(19) United States
(12) Patent Application Publication
(10) Pub. No.: US 2020/0157605 A1
(43) Pub. Date: May 21, 2020

(54) ANALYSIS SYSTEM, ANALYSIS METHOD, AND STORAGE MEDIUM

(71) Applicant: CANON KABUSHIKI KAISHA, Tokyo (JP)

(72) Inventors: Tsutomu Honma, Fuchu-shi (JP); Atsushi Takahashi, Ebina-shi (JP); Masato Minami, Kawasaki-shi (JP); Tetsuya Yano, Tsukuba-shi (JP); Yuki Sato, Nagaoka-shi (JP)

(21) Appl. No.: 16/752,467

(22) Filed: Jan. 24, 2020

Related U.S. Application Data
(63) Continuation of application No. PCT/JP2018/026993, filed on Jul. 19, 2018.

(30) Foreign Application Priority Data
Jul. 27, 2017 (JP) 2017-145585

(51) Int. Cl. C12Q 1/686; G01N 21/64; G06T 7/62; G06F 17/18

(52) U.S. Cl. CPC C12Q 1/686; G01N 21/6486; G06T 7/62; C12Q 2600/158; G06T 2207/10056; G06T 2207/30072; G06F 17/18

(57) ABSTRACT

To analyze the concentration of an analyte in a sample, the number of molecules or particles or concentration of the analyte is determined using a table associating at least one piece of information about a plurality of reaction fields generated by splitting a liquid containing the sample, the at least one piece of information being selected from the group consisting of information about the number of positive reaction fields and information about the number of negative reaction fields, with the number of molecules or particles or concentration of the analyte in the sample or in at least some of the plurality of reaction fields.

### CROSS-REFERENCE TO RELATED APPLICATIONS

This application is a Continuation of International Patent Application No. PCT/JP2018/026993, filed Jul. 19, 2018, which claims the benefit of Japanese Patent Application No. 2017-145585, filed Jul. 27, 2017, both of which are hereby incorporated by reference herein in their entirety.

### TECHNICAL FIELD

The present invention relates to analysis systems, analysis methods, and storage media.

### BACKGROUND ART

Digital polymerase chain reaction (digital PCR, or dPCR) has attracted attention as a method for quantitatively analyzing a nucleic acid having a specific base sequence (target nucleic acid) as an analyte.

In digital PCR, a sample containing a target nucleic acid is mixed with chemicals such as an amplification reagent for amplification of the target nucleic acid and a fluorescent reagent for detection of the target nucleic acid and is diluted and split into a large number of physically independent reaction fields. The sample is diluted so that each reaction field contains either one or no target nucleic acid molecule (such dilution is hereinafter referred to as “limiting dilution”). PCR is then independently performed in each of the plurality of reaction fields to amplify the target nucleic acid and thereby make it detectable. The concentration of the target nucleic acid in the sample can then be determined from the number of reaction fields in which a signal has been detected after amplification (number of positive reaction fields) and/or the number of reaction fields in which no signal has been detected after amplification (number of negative reaction fields).

One method for splitting a reaction solution containing a sample into a large number of physically independent reaction fields in digital PCR is to form droplets of the reaction solution in oil, that is, to form a water-in-oil emulsion (W/O emulsion). This method uses individual droplets in a water-in-oil emulsion as reaction fields (PCT Japanese Translation Patent Publication No. 2012-503773). There are several methods for forming water-in-oil emulsions, including those using microchannel devices and those using mechanical stirring. However, in particular, the formation of a water-in-oil emulsion at high speed tends to cause variation in the size of droplets, that is, the size of reaction fields.

Digital PCR using reaction fields with varying sizes has also been proposed in order to broaden the dynamic range of quantitative analysis (PCT Japanese Translation Patent Publication No. 2014-505476).

One possible method for calculating the concentration or number of molecules or particles of an analyte in reaction fields with varying sizes involves performing calculations by associating information about the size of each reaction field with information about the presence of the analyte in the reaction field. Unfortunately, this method requires very complicated calculations.

In contrast, if the variation in the size of the reaction fields is ignored, that is, if it is assumed that the size distribution of the reaction fields is monodisperse, the concentration or number of molecules or particles of the analyte can be calculated in a simple manner by a known method of calculation as disclosed in PCT Japanese Translation Patent Publication No. 2012-503773. Unfortunately, this method yields analytical results with decreased reliability as the variation in the size of the reaction fields becomes larger.

Accordingly, the present invention provides an analysis system that can yield analytical results with improved reliability in a simpler manner than in the known art even if there is variation in the size of reaction fields.

### SUMMARY OF INVENTION

An analysis system according to one aspect of the present invention is an analysis system for analyzing the concentration of an analyte in a sample. The analysis system includes an analyte-information acquiring section and a determining section. The analyte-information acquiring section is configured to acquire at least one piece of information about a plurality of reaction fields generated by splitting a liquid containing the sample, the at least one piece of information being selected from the group consisting of information about the number of positive reaction fields that are reaction fields in which the analyte is detected and information about the number of negative reaction fields that are reaction fields in which no analyte is detected. The determining section is configured to determine the number of molecules or particles or concentration of the analyte in the sample based on the at least one piece of information acquired by the analyte-information acquiring section, the at least one piece of information being selected from the group consisting of the information about the number of positive reaction fields and the information about the number of negative reaction fields, and a table associating the at least one piece of information selected from the group consisting of the information about the number of positive reaction fields and the information about the number of negative reaction fields with the number of molecules or particles or concentration of the analyte in the sample or in at least some of the plurality of reaction fields.

Further features of the present invention will become apparent from the following description of exemplary embodiments with reference to the attached drawings.

### BRIEF DESCRIPTION OF DRAWINGS

FIG. 1 schematically illustrates the configuration of an analysis system according to a first embodiment.

FIG. 2 schematically illustrates the hardware configuration of an information processing unit.

FIG. 3 is a flowchart showing the steps of an analysis process performed by the analysis system according to the first embodiment.

FIG. 4 schematically illustrates the configuration of an analysis system according to a second embodiment.

FIG. 5 is a flowchart showing the steps of an analysis process performed by the analysis system according to the second embodiment.

FIG. 6 schematically illustrates the configuration of an analysis system according to a third embodiment.

FIG. 7 is a flowchart showing some of the steps of analysis processes performed by the analysis systems according to the third and fourth embodiments.

FIG. 8 schematically illustrates the configuration of an analysis system according to a fourth embodiment.

FIG. 9A is a fluorescence micrograph of Emulsion 1 after thermal cycling.

FIG. 9B is a fluorescence micrograph of Emulsion 2 after thermal cycling.

FIG. 9C is a fluorescence micrograph of Emulsion 3 after thermal cycling.

FIG. 9D is a fluorescence micrograph of Emulsion 4 after thermal cycling.

FIG. 10A is a fluorescence micrograph of Emulsion 5 after thermal cycling.

FIG. 10B is a fluorescence micrograph of Emulsion 6 after thermal cycling.

FIG. 10C is a fluorescence micrograph of Emulsion 7 after thermal cycling.

FIG. 10D is a fluorescence micrograph of Emulsion 8 after thermal cycling.

FIG. 11A is a graph showing the relationship between the relative dilution factor and calculation results of the concentration of an analyte in a sample for Comparative Example 1.

FIG. 11B is a graph showing the relationship between the relative dilution factor and calculation results of the concentration of an analyte in a sample for Example 1.

FIG. 11C is a graph showing the relationship between the relative dilution factor and calculation results of the concentration of an analyte in a sample for Example 2.

FIG. 12A is a graph showing the relationship between the relative dilution factor and calculation results of the concentration of an analyte in a sample for Comparative Example 2.

FIG. 12B is a graph showing the relationship between the relative dilution factor and calculation results of the concentration of an analyte in a sample for Example 3.

FIG. 12C is a graph showing the relationship between the relative dilution factor and calculation results of the concentration of an analyte in a sample for Example 4.

### DESCRIPTION OF EMBODIMENTS

Embodiments of the present invention will hereinafter be described with reference to the drawings. It should be understood that the invention is not limited to the following embodiments. Rather, for example, modifications and improvements may be made to the following embodiments as appropriate based on the ordinary knowledge of those skilled in the art without departing from the spirit of the invention, and such modifications and improvements are also included within the scope of the invention.

### First Embodiment

An analysis system according to a first embodiment of the present invention will now be described with reference to the drawings.

Configuration of Analysis System

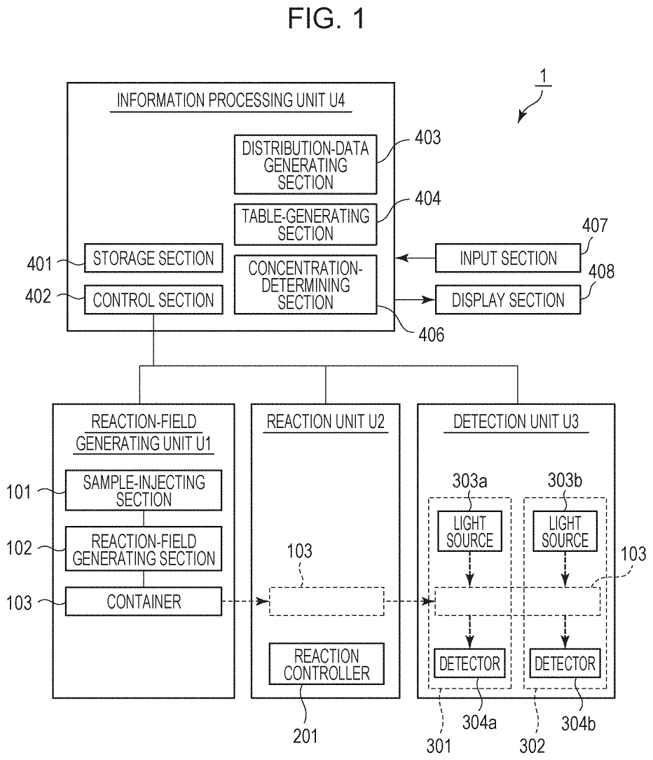

FIG. 1 schematically illustrates the configuration of the analysis system according to this embodiment. An analysis system 1 according to this embodiment includes a reaction-field generating unit U1, a reaction unit U2, a detection unit U3, and an information processing unit U4. The individual units may be connected in part or in whole via a network such as a LAN or the Internet.

Reaction-Field Generating Unit

The reaction-field generating unit U1 is a unit configured to split a liquid such as a reaction solution containing an analyte in a sample to generate a plurality of reaction fields that are physically independent of each other.

Reaction Field

As used herein, the term “reaction field” refers to a space surrounded by at least one interface selected from the group consisting of liquid-liquid interfaces, gas-liquid interfaces, and solid-liquid interfaces. A reaction in a reaction field occurs within its closed space and proceeds independently of other reaction fields. In other words, a reaction in one reaction field involves only substances confined within the space defined by the interface described above.

For example, if the reaction solution is dispensed into each of a plurality of wells of a plate such as a microplate, the reaction solution dispensed into each well serves as a reaction field. In this case, the reaction field is surrounded by the solid-liquid interface between the wall surface of the well and the reaction solution and the gas-liquid interface between air and the reaction solution. Alternatively, if the reaction solution forms droplets in an emulsion such as a water-in-oil emulsion (W/O emulsion), each droplet in the emulsion serves as a reaction field. In this case, the reaction field is surrounded by the liquid-liquid interface between the continuous phase and the dispersed phase.

Analyte

As used herein, the term “analyte” refers to a compound or particle that is present in a sample and that is of interest in quantitative analysis. In this embodiment, the analyte may be any substance that can be made detectable by a reaction in a reaction field, described later. Examples of analytes include nucleic acids, peptides, proteins, and enzymes.

As used herein, the term “make detectable” refers to making a signal derived from the analyte detectable by a reaction in the reaction unit U2, described later. For example, a signal that is originally undetectably weak can be amplified and made detectable by increasing the number of molecules or particles or concentration of the analyte by an amplification reaction in the reaction unit U2. The analyte can also be made detectable by generating a substance that generates a certain signal from the analyte by a reaction. Alternatively, the analyte can be made detectable by changing the analyte so as to generate a signal, for example, by a chemical change.

Nucleic acids, as described in detail later, can be made detectable by a nucleic acid amplification reaction such as PCR using, as chemicals for making nucleic acids detectable, an amplification reagent for amplification of nucleic acids and a fluorescent reagent that emits fluorescence by interaction with nucleic acids. Peptides and proteins can be made detectable, for example, by enzyme-linked immunosorbent assay (ELISA). The analyte may also be a substance including, for example, a nucleic acid, a peptide, or a protein. For example, the analyte may be a molecule, microparticle, nanoparticle, or cell having a nucleic acid, a peptide, a protein, or any combination thereof adhering thereto or attached thereto by a covalent bond or other bond.

For example, if the specimen is blood collected from a human or a nucleic acid extracted therefrom, and the analyte is a nucleic acid that can be present in the specimen and that contains a gene associated with a disease such as cancer or an infectious disease, information useful for purposes such as disease diagnosis can be expected to be available. If the specimen is food, food inspection such as the assessment of genetically modified organisms (GMOs) can be performed. Alternatively, if the specimen is soil or water in an environment, environmental monitoring can be performed.

If the analyte in this embodiment is a nucleic acid, the nucleic acid may be any nucleic acid that serves as a template nucleic acid for amplification and may be either deoxyribonucleic acid (DNA) or ribonucleic acid (RNA). The nucleic acid may take any form and may be a linear nucleic acid or a cyclic nucleic acid. The nucleic acid may be one type of nucleic acid having a single base sequence or may be a plurality of types of nucleic acids having various base sequences (e.g., a complementary DNA library).

Specimen

As used herein, the term “specimen” refers to a sample source collected or extracted from, for example, an organism, food, or an environment. In general, the concentration of an analyte in a sample subjected to quantitative analysis is converted to the concentration in the specimen for various purposes such as medical diagnosis and food and environmental assessment.

Sample

As used herein, the term “sample” refers to a material to be subjected to analysis according to this embodiment. In this embodiment, the concentration of an analyte in a sample is measured. The sample may be a specimen itself or may be a specimen subjected to pretreatment or adjustment for analysis, such as purification, concentration, and the chemical modification and fragmentation of the analyte. The concentration of the analyte in the sample (the number of molecules or particles per unit volume) may be, but not limited to, a level at which, when a plurality of reaction fields are generated, each of the plurality of reaction fields contains one or no analyte molecule or particle. This can improve the reliability of analytical results.

The reaction-field generating unit U1 includes a sample-injecting section 101, a reaction-field generating section 102, and a container 103.

Sample-Injecting Section

The sample-injecting section 101 is a section configured to inject a reaction solution that is a liquid containing a sample into the reaction-field generating section 102.

The reaction solution injected from the sample-injecting section 101 is fed to the reaction-field generating section 102. The reaction solution may be fed by a feed unit (not shown) such as a pump. The reaction solution injected from the sample-injecting section 101 may also be mixed with an oil serving as a continuous phase for forming an emulsion while being fed to the reaction-field generating section 102. Alternatively, the sample alone may be injected from the sample-injecting section 101 and may then be mixed with other materials such as chemicals for analyte detection to generate a reaction solution while being fed to the reaction-field generating section 102.

Reaction Solution

As used herein, the term “reaction solution” refers to a liquid containing at least a sample containing an analyte and a chemical for making the analyte detectable. The reaction solution may be an aqueous liquid containing water.

Chemical for Making Analyte Detectable

If the analyte is a nucleic acid, the analyte can be made detectable by amplifying the nucleic acid using a nucleic acid amplification reaction in the presence of an enzyme, such as PCR. Here, examples of nucleic acid amplification reactions that can be used include those in which a reaction is allowed to proceed by subjecting a reaction field to thermal cycling, such as PCR and ligase chain reaction (LCR), and those in which a reaction is allowed to proceed by temperature control without subjecting a reaction field to thermal cycling, such as strand displacement amplification (SDA), isothermal and chimeric primer-initiated amplification of nucleic acids (ICAN), and loop-mediated isothermal amplification (LAMP).

If a nucleic acid amplification reaction is used, an amplification reagent for amplification of the nucleic acid and a fluorescent reagent that emits fluorescence by interaction with the nucleic acid are used as chemicals for making the nucleic acid detectable.

The amplification reagent contains one or a pair of primers (forward and reverse primers) having a base sequence complementary to a predetermined base sequence of the target nucleic acid serving as the analyte and a polymerase serving as a biological catalyst to promote a nucleic acid synthesis reaction. The polymerase is preferably a heat-resistant polymerase, more preferably a heat-resistant DNA polymerase. The amplification reagent further contains a ribonucleic acid such as deoxyribonucleotide 5′-triphosphate (dNTP) as a raw material for the nucleic acid. The amplification reagent may further contain a buffer or buffer solution and a salt for control of the hydrogen ion concentration (pH) of the reaction solution. The amplification reagent may be a commercially available kit containing the above components.

The primers may be any oligonucleotide that hybridizes to the base sequence of a portion of the target nucleic acid under stringent conditions and that can be used for a nucleic acid amplification reaction. Here, the stringent conditions are those under which a primer having at least 90% or more sequence identity, preferably 95% or more sequence identity, with the template nucleic acid can hybridize specifically to the template nucleic acid. The primers can be designed as appropriate based on the base sequence of the target nucleic acid. It is desirable to design the primers depending on the type of nucleic acid amplification. The primers typically have a length of 5 to 50 nucleotides, preferably 10 to 40 nucleotides. The primers can be generated by a nucleic acid synthesis method that is commonly used in the field of molecular biology.

The buffer or buffer solution may be any suitable buffer or buffer solution. The buffer or buffer solution may be configured to maintain the hydrogen ion concentration (pH) of the reaction solution at or near a pH at which the desired reaction can occur efficiently. If PCR is performed, the pH of the reaction solution can be freely selected depending on the components of the amplification reagent used, for example, in the range of 6.5 to 9.0. The buffer or buffer solution may be a buffer or buffer solution that is commonly used in the field of molecular biology. Examples of buffers that can be used include tris(hydroxymethyl)aminomethane (Tris) buffer, 4-(2-hydroxyethyl)-1-piperazineethanesulfonic acid (HEPES) buffer, and 2-morpholinoethanesulfonic acid (MES) buffer.

The salt used may be selected as appropriate from, for example, CaCl₂, KCl, MgCl₂, MgSO₄, NaCl, and combinations thereof.

The fluorescent reagent is a chemical that emits fluorescence by interaction with the nucleic acid. The fluorescent reagent may be a fluorescent reagent that is commonly used for PCR, such as a fluorescent intercalator (fluorescent dye) or a probe for probe assays (fluorescent labeled probe). Examples of suitable fluorescent intercalators that can be used include ethidium bromide, SYBR Green I (“SYBR” is a registered trademark of Molecular Probes, Inc.), and LC Green. The fluorescent labeled probe may be an oligonucleotide (probe) that hybridizes specifically to the target nucleic acid and that has one end (5′-end) modified with a reporter and the other end (3′-end) modified with a quencher. Examples of reporters that can be used include fluorescent substances such as fluorescein 5-isothiocyanate (FITC) and VIC. Examples of quenchers that can be used include fluorescent substances such as TAMARA and other substances such as Eclipse, DABCYL, and MGB. Examples of fluorescent labeled probes that can be used include TaqMan probes (“TaqMan” is a registered trademark of Roche Diagnostics). Although an example in which fluorescent reagents are used has been described here, luminescent reagents that emit luminescence other than fluorescence may also be used.

On the other hand, if the analyte is a peptide or protein, the analyte can be made detectable by an antigen-antibody reaction with an antibody (or antigen) that reacts specifically with the analyte and an enzymatic reaction in the presence of an enzyme, such as ELISA. More specifically, for example, the analyte is combined with an enzyme-labeled antibody (or antigen) by an antigen-antibody reaction, and a chromogenic or luminescent substance resulting from an enzymatic reaction in the presence of the enzyme is detected. The antibody (or antigen) that undergoes the antigen-antibody reaction with the analyte need not be enzyme-labeled in advance, but may be enzyme-labeled after the antigen-antibody reaction.

If ELISA is used, a reagent containing an antibody (or antigen) and an enzyme is used as a chemical for making the analyte detectable. The reagent used for ELISA may be a commercially available kit.

If a plurality of chemicals that make different analytes detectable so that they can be distinguished are used, for example, if the resulting fluorescence has different wavelengths, a plurality of analytes can be simultaneously detected by one analysis.

Reaction-Field Generating Section

The reaction-field generating section 102 splits the reaction solution injected from the sample-injecting section 101 to generate a plurality of reaction fields that are physically independent of each other. Examples of methods for splitting the reaction solution to generate a plurality of reaction fields include the following methods.

The first method is to dispense the reaction solution into each of a plurality of extremely small wells formed on a glass or resin substrate such as a microwell plate. Thus, the interior of each of the extremely small wells serves as a reaction field.

The second method is to apply the reaction solution to a surface of a glass or resin substrate subjected to water-repellent treatment or oil-repellent treatment in a predetermined pattern. For example, if an aqueous reaction solution is applied to a glass substrate subjected to water-repellent treatment in a grid pattern, droplets are formed inside the individual squares. Each of the plurality of droplets serves as a reaction field.

The third method is to form, from the reaction solution and a liquid incompatible with the reaction solution (hereinafter referred to as “incompatible liquid”), an emulsion of the reaction solution dispersed in droplet form in the incompatible liquid. In other words, this method is to form an emulsion in which the incompatible liquid is a continuous phase and the reaction solution is a dispersed phase. For example, if a reaction solution that is an aqueous liquid containing water and an oily liquid (oil) are mixed together to form a water-in-oil emulsion (W/O emulsion), each of the droplets of the reaction solution dispersed in the oil serves as a reaction field.

Of these, the reaction-field generating section 102 may generate reaction fields by the third method, that is, by forming, from the reaction solution and an incompatible liquid, an emulsion of the reaction solution dispersed in droplet form in the incompatible liquid. That is, the reaction-field generating section 102 may be an emulsion-generating section configured to generate an emulsion from the reaction solution and an incompatible liquid incompatible with the reaction solution.

Emulsion-Generating Section

The emulsion may be generated by any process, including conventionally known emulsification processes. One example is mechanical emulsification, in which an emulsion is formed by applying mechanical energy using a device such as an agitator or ultrasonic homogenizer. Other examples include processes using microchannel devices, such as microchannel emulsification and crossflow microchannel emulsification, and membrane emulsification using emulsification membranes. These processes may be used alone or in combination. Of these, mechanical emulsification and membrane emulsification are preferred because an emulsion can be formed at high throughput, although they tend to result in larger variation (variance) in the size of droplets than processes using microchannel devices. Membrane emulsification is particularly preferred because, for example, it can simplify the device configuration of the device for forming the emulsion and can form an emulsion with relatively small variation in the size of droplets. That is, the reaction-field generating section 102 is more preferably a membrane emulsification unit or a mechanical emulsification unit, particularly preferably a membrane emulsification unit.

Membrane emulsification is the process of forming an emulsion by allowing a dispersed phase or a continuous phase, or a mixture of a dispersed phase and a continuous phase, to pass through an emulsification membrane having a plurality of pores or slits. In membrane emulsification, a dispersed phase or a continuous phase, or a mixture of a dispersed phase and a continuous phase, may be allowed to pass through an emulsification membrane any number of times, and it may be performed either once or multiple times.

Examples of membrane emulsification processes that can be used include direct membrane emulsification and pumping emulsification. Direct membrane emulsification is the process of forcing a dispersed phase through an emulsification membrane under a certain pressure to form an emulsion with a continuous phase flowing slowly on the side where the dispersed phase is forced out of the emulsification membrane. Pumping emulsification is the process of alternately forcing a continuous phase and a dispersed phase out of two syringes filled therewith to pass through an emulsification membrane held between the two syringes, thereby forming an emulsion. In pumping emulsification, one of the two syringes may be filled with a mixture of a continuous phase and a dispersed phase, with the other syringe being empty. In pumping emulsification, a pumping-type emulsification device including an emulsification membrane held between a pair of connectors connectable to syringes can be used.

The emulsification membrane used for membrane emulsification may be a porous membrane having a plurality of pores or a membrane having slits. Specific examples of emulsification membranes that can be used include porous glass membranes such as shirasu porous glass (SPG) membranes, polycarbonate membrane filters, and polytetrafluoroethylene (PTFE) membrane filters. The surface of the emulsification membrane may be subjected to hydrophobic treatment. The pore diameter of the emulsification membrane can be selected depending on the size of droplets in the water-in-oil emulsion to be formed, preferably from 0.2 μm to 100 μm, more preferably from 5 μm to 50 μm.

Oil

If the reaction solution is an aqueous liquid containing water, an oily liquid (oil) can be used as an incompatible liquid incompatible with the reaction solution. In this case, the reaction-field generating section 102 generates a W/O emulsion.

Examples of oils that can be used include hydrocarbon oils, silicone oils, and fluorinated oils. Examples of hydrocarbon oils that can be used include mineral oils; animal- and plant-based oils such as squalane oil and olive oil; paraffinic hydrocarbons having 10 to 20 carbon atoms, such as n-hexadecane; and olefinic hydrocarbons having 10 to 20 carbon atoms. Examples of commercially available hydrocarbon oils that can be used include TEGOSOFT DEC (diethylhexyl carbonate) (available from Evonik; “TEGOSOFT” is a registered trademark of Evonik). Examples of fluorinated oils that can be used include HFE-7500 (2-(trifluoromethyl)-3-ethoxydodecafluorohexane). Examples of commercially available fluorinated oils that can be used include FLUORINERT FC-40 and FLUORINERT FC-3283 (available from 3M; “FLUORINERT” is a registered trademark of 3M). Hydrocarbon oils, silicone oils, and fluorinated oils may also be used in combination as appropriate.

Other Additives

A surfactant may be further added when an emulsion is generated. The addition of a surfactant can be expected to be effective in, for example, controlling the size of the droplets in the emulsion and stably maintaining the emulsion. The surfactant may be a conventionally known surfactant that is commonly used in emulsification processes, for example, a nonionic surfactant, a fluorinated resin, or a phosphocholine-containing resin. Examples of nonionic surfactants that can be used include hydrocarbon surfactants, silicone surfactants, and fluorinated surfactants.

Examples of commercially available hydrocarbon nonionic surfactants that can be used include Pluronic F-68 (polyoxyethylene-polyoxypropylene block copolymer) (available from Sigma-Aldrich; “Pluronic” is a registered trademark of BASF), Span 60 (sorbitan monostearate) (available from Tokyo Chemical Industry Co., Ltd.; “Span” is a registered trademark of Croda International plc), Span 80 (sorbitan monooleate) (available from Sigma-Aldrich; “Span” is a registered trademark of Croda International plc), Triton-X100 (polyoxyethylene(10) octylphenyl ether) (available from Sigma-Aldrich; “Triton” is a registered trademark of Union Carbide), and Tween 20 (polyoxyethylene sorbitan monolaurate) and Tween 80 (polyoxyethylene sorbitan monooleate) (both available from Sigma-Aldrich; “Tween” is a registered trademark of Croda International plc). Examples of silicone nonionic surfactants that can be used include ABIL EM90 (cetyl dimethicone copolyol (cetyl PEG/PPG-10/1 dimethicone)), ABIL EM 120 (bis-(glyceryl/lauryl) glyceryl lauryl dimethicone), ABIL EM 180 (cetyl PEG/PPG-10/1 dimethicone), and ABIL WE 09 (polyglyceryl-4 isostearate, cetyl dimethicone copolyol, and hexyl laurate) (all available from Evonik; “ABIL” is a registered trademark of Evonik). Examples of fluorinated resins that can be used include Krytox-AS (“Krytox” is a registered trademark of Chemours). Examples of phosphocholine-containing resins that can be used include Lipidure-S (available from NOF Corporation; “Lipidure” is a registered trademark of NOF Corporation).

The concentration of the surfactant in the emulsion is preferably, but not limited to, from 0.01% by mass to 10% by mass, more preferably from 0.1% by mass to 8% by mass, even more preferably from 1% by mass to 4% by mass.

The volume ratio of the incompatible liquid (continuous phase) to the reaction solution (dispersed phase) in the emulsion is preferably, but not limited to, from 1 to 300, more preferably from 1 to 150.

The size of the droplets in the emulsion is preferably, but not limited to, from 1 μm to 300 μm in diameter, more preferably from 1 μm to 200 μm in diameter, even more preferably from 20 μm to 150 μm in diameter. If the diameter of the droplets is 300 μm or less, a large number of droplets (reaction fields) can be formed even if the volume of the specimen or sample is as small as tens to hundreds of microliters, as in clinical examination, thus improving the analysis accuracy. In addition, if the diameter of the droplets is 300 μm or less, the stability of the emulsion can be improved.

The size distribution of the droplets in the emulsion may be polydisperse. As used herein, the term “polydisperse” refers to not being monodisperse, that is, not being uniform but varying in the size of the droplets. The droplets in the emulsion may have a nearly monodisperse distribution (e.g., the coefficient of variation (CV) of the diameter of the droplets is several percent or less) or may be a mixture of droplets with different sizes. In general, the droplets in an emulsion generated by mechanical emulsification or membrane emulsification often have a size distribution in which the coefficient of variation (CV) of the diameter of the droplets is about 10% to about 20%, or higher. In particular, the size of the droplets in the emulsion tends to be polydisperse when the emulsion is formed at high speed. According to this embodiment, quantitative analysis can be performed with high reliability even if there is variation in the size of the droplets.

The emulsion preferably contains from 100 to 1,000,000,000 droplets, more preferably from 100 to 20,000,000 droplets, even more preferably from 2,000 to 20,000,000 droplets. As described later, according to estimations conducted by the inventors, it may be determined that at least 100 or more droplets contain the analyte to ensure sufficient reliability of analytical results in digital analysis. Accordingly, the emulsion may contain 100 or more droplets. For example, in general, the volume of a reaction solution in clinical examination is often set to about 0.01 mL to about 0.5 mL. If the size of the droplets is about 10 μm to about 200 μm, the number of the droplets is set to about 2,000 to about 1,000,000,000 droplets.

Container

The container 103 is a container configured to retain a plurality of reaction fields generated by the reaction-field generating section 102. If the reaction-field generating section 102 is an emulsion-generating section, the container 103 is a container configured to contain an emulsion generated by the emulsion-generating section. If the reaction-field generating section 102 generates a plurality of reaction fields by dispensing the reaction solution into each of a plurality of extremely small wells formed on a glass or resin substrate, the substrate having the wells formed thereon serves as the container 103.

While retaining the plurality of reaction fields, the container 103 is transported from the reaction-field generating unit U1 to the reaction unit U2 and then to the detection unit U3 by a transport unit (not shown). Although an example in which the container 103 is transported between the units by the transport unit (not shown) is described, the container 103 is not limited thereto, but may be transported by an operator of the analysis system 1.

The container 103 may be configured to be attachable to and detachable from the reaction-field generating unit U1 together with part or the entirety of the sample-injecting section 101 and the reaction-field generating section 102. That is, the reaction-field generating unit U1 may be configured such that a cartridge having the functions of the sample-injecting section 101, the reaction-field generating section 102, and the container 103 is attachable to and detachable from the main body of the reaction-field generating unit U1. This configuration can prevent contamination between samples. The cartridge may include a reaction controller 201, described later.

Reaction Unit

The reaction unit U2 is a unit including the reaction controller 201 and configured to allow a reaction to proceed in each of the plurality of reaction fields generated by the reaction-field generating unit U1. By this reaction, the analyte present in each of the plurality of reaction fields can be made detectable.

Reaction

If the analyte is a nucleic acid, as described above, the analyte can be made detectable by amplifying the nucleic acid using a nucleic acid amplification reaction in the presence of an enzyme, such as PCR. As described above, examples of nucleic acid amplification reactions that can be used include PCR, LCR, SDA, ICAN, and LAMP. If such a nucleic acid amplification reaction is performed, the reaction may be controlled by regulating the temperature of the reaction fields, for example, by subjecting the reaction fields to thermal cycling, by maintaining the reaction fields at a certain temperature, or by applying a predetermined temperature profile. An alternative known method for controlling the reaction involves allowing the reaction fields to flow through microchannels, having a predetermined shape, of a microchannel device (Science, 280, 1046 (1998)).

If the analyte is a peptide or protein, as described above, the analyte can be made detectable by a molecular biology approach combining an antigen-antibody reaction with an enzymatic reaction, such as ELISA. In this case, the temperature of the reaction fields may be regulated to maintain the temperature of the reaction fields at a predetermined temperature.

Reaction Controller

The reaction controller 201 controls the reaction in each of the plurality of reaction fields in the container 103. The reaction controller 201 can also be regarded as a reaction section configured to allow a reaction to proceed in each of the plurality of reaction fields in the container 103. The reaction controller 201 may control the reaction in each of the plurality of reaction fields in any manner. For example, the reaction controller 201 may control the reaction by controlling the temperature of the reaction fields or may control the reaction by controlling the position or speed of the reaction fields in microchannels. That is, the reaction controller 201 may include a temperature regulator such as a heater or cooler or may include a pump connected to microchannels.

The reaction controller 201 may also apply at least one selected from the group consisting of heat, magnetic field, electric field, current, light, and radiation to each of the plurality of reaction fields depending on the type of reaction in the reaction fields. The reaction unit U2 may be a commercially available thermal cycler.

Detection Unit

The detection unit U3 is a unit configured to detect the size of each of the plurality of reaction fields and to detect the analyte in each of the plurality of reaction fields. The detection unit U3 includes an analyte-information acquiring section 301 configured to acquire information about the presence of the analyte in each of the plurality of reaction fields and a size-information acquiring section 302 configured to acquire information about the size of each of the plurality of reaction fields.

Although the detection unit U3 may select some of the plurality of reaction fields retained in the container 103 for detection, the detection unit U3 may perform detection on all reaction fields available for measurement. This can increase the number of reaction fields subjected to detection and can thus improve the reliability of analytical results.

Analyte-Information Acquiring Section

The analyte-information acquiring section 301 is a section configured to detect the analyte in each of the plurality of reaction fields. The analyte-information acquiring section 301 detects a signal derived from the analyte in each of the plurality of reaction fields in which reactions have proceeded in the reaction unit U2. The analyte-information acquiring section 301 detects a signal derived from the analyte to determine whether the analyte is present in each of the plurality of reaction fields. In this way, the analyte-information acquiring section 301 acquires information about the presence of the analyte in each of the plurality of reaction fields (analyte information). Light is suitable for use as a signal.

A reaction field in which a signal has been detected by the analyte-information acquiring section 301, that is, a reaction field containing the analyte, is herein referred to as “positive reaction field”. A reaction field in which no signal has been detected by the analyte-information acquiring section 301, that is, a reaction field containing no analyte, is herein referred to as “negative reaction field”. It is assumed herein that no signal has been detected if the strength of the signal detected by the analyte-information acquiring section 301 is weaker than a predetermined threshold. That is, it is determined whether the analyte is present in a reaction field by comparing the strength of the signal from the reaction field with a predetermined threshold.

For example, if the analyte is a nucleic acid, and an amplification reagent and a fluorescent reagent are used as chemicals for making the analyte detectable, the analyte-information acquiring section 301 may detect fluorescence at a predetermined wavelength as a signal derived from the analyte.

If the analyte-information acquiring section 301 detects light such as fluorescence as a signal, the analyte-information acquiring section 301 may include a light source 303a, a detector 304a, and a controller (not shown). The light source 303a irradiates each of the plurality of reaction fields retained in the container 103 with light at the wavelength depending on the signal to be detected. The detector 304a detects a signal generated from each of the plurality of reaction fields irradiated with the light. That is, the light source 303a functions as an excitation unit, whereas the detector 304a functions as a light detection unit.

The detector 304a may be, for example, a photodiode, a line sensor, or an image sensor (image capture device). In particular, an image sensor may be used because it can simultaneously detect signals from many reaction fields. Examples of image sensors that can be used include charge-coupled device (CCD) image sensors and complementary metal oxide semiconductor (CMOS) image sensors. Alternatively, the detector 304a may be a digital camera including an image sensor. If the detector 304a detects light, an optical filter may be used to adjust the wavelength of the light from the reaction fields.

The analyte-information acquiring section 301 may be a flow cytometer, which sequentially performs detection on a plurality of reaction fields flowing through a channel. Alternatively, the analyte-information acquiring section 301 may two-dimensionally perform excitation and detection on a plurality of reaction fields. That is, the analyte-information acquiring section 301 may be configured to two-dimensionally perform excitation by irradiating a plurality of reaction fields arranged in a plane with light and to two-dimensionally detect signals generated from the reaction fields using an image sensor. This configuration allows signals from many reaction fields to be detected at high throughput.

The light source 303a of the analyte-information acquiring section 301 may be a light source configured to irradiate the reaction fields with light at different wavelengths. For example, the light source 303a may be a variable-wavelength light source or may include a plurality of light sources that emit light at different wavelengths. In this case, if a plurality of chemicals that make different analytes detectable so that they can be distinguished are used, for example, if the resulting fluorescence has different wavelengths, a plurality of analytes can be simultaneously detected by one analysis.

Size-Information Acquiring Section

The size-information acquiring section 302 is a section configured to detect the size of each of the plurality of reaction fields. The size-information acquiring section 302 detects the size of each of the plurality of reaction fields in which reactions have proceeded in the reaction unit U2.

The size-information acquiring section 302 may include a detector 304b and a controller (not shown). The detector 304b detects light from the reaction fields. The detector 304b may be a detector similar to the detector 304a. Alternatively, the detector 304a may function as the detector 304b. The size-information acquiring section 302 may further include a light source 303b. The light source 303b may be a light source that emits light at a different wavelength from the light source 303a. If the light source 303b is a variable-wavelength light source, the light source 303b may function as the light source 303a.

The size-information acquiring section 302 may detect the size of the reaction fields by detecting scattered light from the reaction fields. The detector 304b of the size-information acquiring section 302 may be an image sensor because it can simultaneously detect many reaction fields.

In particular, the size-information acquiring section 302 may acquire an image of many reaction fields present in a field of view, and the controller (not shown) may acquire the size of the reaction fields from the image data. In this case, for example, the acquired image data may be analyzed with commonly used image analysis software to acquire information about the size of each reaction field. For example, if the reaction fields are droplets that can be regarded as perfect spheres, at least one piece of information selected from the group consisting of the radius, diameter, cross-sectional area, and volume of the droplets can be acquired from the image.

Information Processing Unit

The information processing unit U4 is a unit configured to determine the number of molecules or particles or concentration of the analyte in the sample based on detection results from the detection unit U3 (information about the size of each of the reaction fields and information about the presence of the analyte in each of the reaction fields).

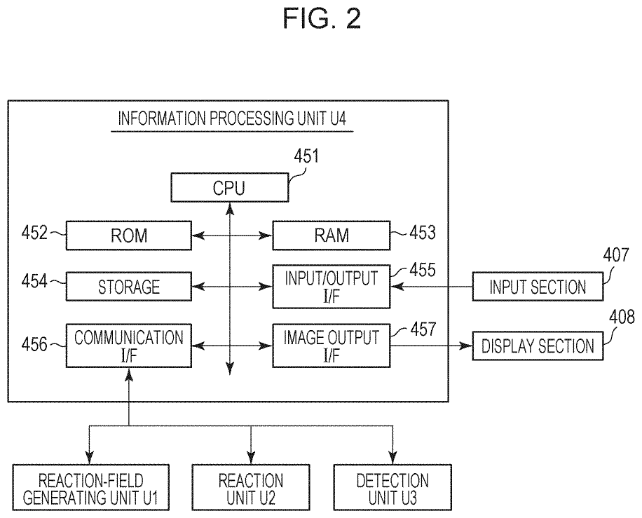

FIG. 2 is a hardware diagram of the information processing unit U4. The information processing unit U4 includes, as its hardware, a CPU 451, a ROM 452, a RAM 453, a storage 454, an input/output I/F 455, a communication I/F 456, and an image output I/F 457.

The CPU 451 executes a program stored in the ROM 452 or a program loaded into the RAM 453 to control the various sections of the information processing unit U4. The ROM 452 is a nonvolatile memory and stores, for example, a program necessary for initial operation of the information processing unit U4. The RAM 453 is a volatile memory and is used to read a program from the ROM 452, the storage 454, or an external storage device (not shown). The RAM 453 is also used as a working space when the CPU 451 executes such programs.

The storage 454 has installed therein various programs for execution by the CPU 451, such as an operating system and application programs, as well as data used for execution of the programs. The storage 454 also has installed therein a program for analyzing measurement data received from the detection unit U3 and outputting analytical results.

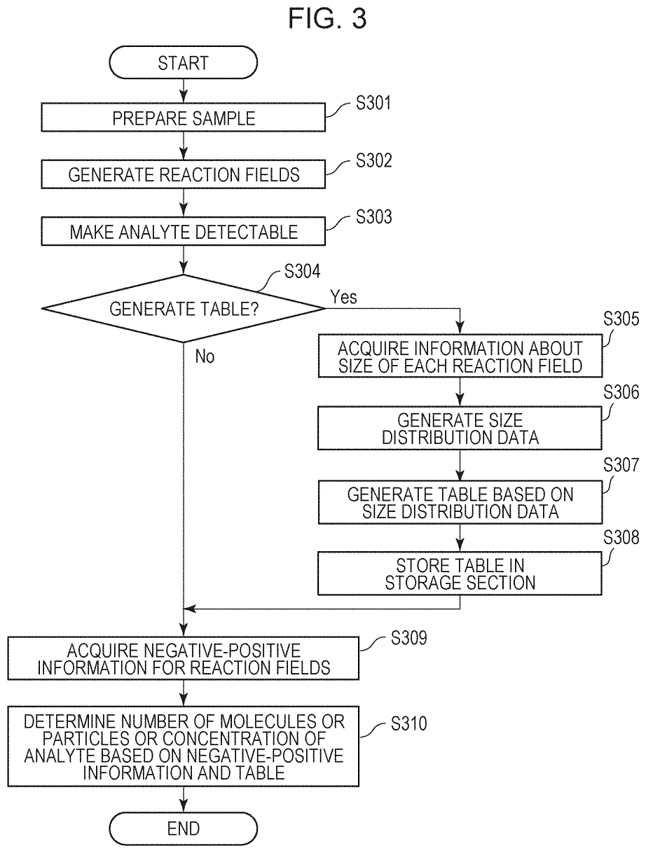

A program stored in a storage medium such as the storage 454 is loaded into the RAM 453, and the CPU 451 operates according to the program loaded into the RAM 453 to execute the functions of the various sections shown in FIG. 1 and the various steps shown in FIG. 3, described later.

The input/output interface (I/F) 455 is connected to an input section 407 including, for example, a mouse, a keyboard, and a touch panel. A user uses the input section 407 to input data into the information processing unit U4. The image output interface (I/F) 457 is connected to a display section 408 including, for example, a liquid crystal panel. The image output interface (I/F) 457 outputs a video signal corresponding to image data to the display section 408. The display section 408 displays an image based on the input video signal. The information processing unit U4 is also connected via the communication interface (I/F) 456 to the reaction-field generating unit U1, the reaction unit U2, the detection unit U3, and the transport unit (not shown). The communication interface 456 allows the information processing unit U4 to communicate data with the various units described above.

As shown in FIG. 1, the information processing unit U4 includes, as its functions, a storage section 401, a control section 402, a distribution-data generating section 403, a table-generating section 404, and a concentration-determining section 406.

The control section 402 is a section configured to control the operation of the reaction-field generating unit U1, the reaction unit U2, the detection unit U3, and the transport unit (not shown).

Storage Section

The storage section 401 is a section configured to store data received from the detection unit U3 or the input section 407 and data generated by various procedures performed by the information processing unit U4. In this embodiment, the storage section 401 is configured to store a table associating at least one piece of information selected from the group consisting of information about the number of positive reaction fields and information about the number of negative reaction fields with information about the number of molecules or particles of the analyte. As used herein, the term “information about the number” encompasses, for example, the number itself and the proportion thereof. In the following description, “at least one piece of information selected from the group consisting of information about the number of positive reaction fields and information about the number of negative reaction fields” may also be referred to as “negative-positive information”. The data received from the detection unit U3 or other unit and containing the negative-positive information may also be referred to as “detection data”.

The table stored in the storage section 401 is a table associating the negative-positive information acquired by the analyte-information acquiring section 301 with the number of molecules or particles or concentration of the analyte, as described above, and can also be termed calibration curve information. As used herein, the number of molecules or particles or concentration of the analyte may be the number of molecules or particles or concentration of the analyte in the sample, the number of molecules or particles or concentration of the analyte in the reaction solution, or the number of molecules or particles or concentration of the analyte in at least some of the plurality of reaction fields.

The table stored in the storage section 401 may be a table theoretically generated based on information about the size of each of the plurality of reaction fields or may be a table experimentally acquired from standard samples with known analyte concentrations. The table stored in the storage section 401 may be a table theoretically generated based on information about the size of each of the plurality of reaction fields because such a table can be acquired without providing standard samples and can reflect the size distribution of the reaction fields.

The table stored in the storage section 401 may be a table generated by the table-generating section 404, described later, or may be a table generated by a device other than the analysis system 1. If the table is generated by a device other than the analysis system 1, the information processing unit U4 can acquire the table via the communication I/F 456 or other interface and can store the table in the storage section 401.

Distribution-Data Generating Section

The distribution-data generating section 403 receives information about the size of each of the reaction fields from the detection unit U3 and generates distribution data. More specifically, the distribution-data generating section 403 divides the size distribution of the reaction fields into a plurality of classes (bins) and generates distribution data (size distribution data) containing information about the number of reaction fields for each class.

For example, it is supposed that each of the plurality of reaction fields is substantially spherical, and the minimum and maximum sizes of the reaction fields in terms of equivalent spherical diameter are 10 μm and 210 μm, respectively. In this case, for example, the distribution-data generating section 403 divides the size distribution of the reaction fields into ten classes at intervals of 20 μm and counts the number of reaction fields with sizes that fall within each class. The distribution-data generating section 403 may generate distribution data with any number of classes and any class interval, which may be determined depending on the resolution of the size-information acquiring section 302 or may be determined by a method that is commonly used in statistical processing.

Table-Generating Section

The table-generating section 404 generates, based on the size information acquired by the size-information acquiring section 302, a table used for the determination of the number of molecules or particles or concentration of the analyte by the concentration-determining section 406. The table generated by the table-generating section 404 is stored in the storage section 401 and is used for the determination of the number of molecules or particles or concentration of the analyte by the concentration-determining section 406.

The table-generating section 404 may generate a table based on the size information acquired by the size-information acquiring section 302 by assuming the number of molecules or particles of the analyte and performing a simulation based on the Poisson model. A specific example of the method for table generation by the table-generating section 404 includes steps (1) to (6) below:

(1) The distribution data is received from the distribution-data generating section 403.

(2) The number of molecules or particles of the analyte present, before the generation of the plurality of reaction fields, in the liquid corresponding to the plurality of reaction fields subjected to detection for the acquisition of the distribution data (setting value 1) is assumed.

(3) The assumed analyte is sorted into each of the plurality of classes in the distribution data. Here, the analyte is sorted into each class in proportion to the proportion of the total volume of the reaction fields in the class to the total volume of all reaction fields.

(4) The number of reaction fields containing the analyte and the number of reaction fields containing no analyte in each of the plurality of classes in the distribution data are probabilistically calculated based on the Poisson model. The number of reaction fields containing the analyte is referred to as “number of positive reaction fields”, whereas the number of reaction fields containing no analyte is referred to as “number of negative reaction fields”. For example, the proportion of the reaction fields containing no analyte molecule or particle in each of the plurality of classes may be calculated based on the Poisson model, and the number of negative reaction fields and the number of positive reaction fields may be calculated based on the calculated proportion.

(5) The numbers of positive reaction fields calculated for the individual classes are added together to calculate the number of positive reaction fields in all reaction fields, and the numbers of negative reaction fields calculated for the individual classes are added together to calculate the number of negative reaction fields in all reaction fields. Thus, the number of positive reaction fields and the number of negative reaction fields are associated with the number of molecules or particles of the analyte (setting value 1). Alternatively, the proportion of the positive reaction fields and the proportion of the negative reaction fields may be calculated and associated with the number of molecules or particles of the analyte (setting value 1).

(6) Steps (2) to (5) are repeated with varying setting values to associate each of n setting values (setting values 1 to n) with at least one piece of information selected from the group consisting of information about the number of positive reaction fields and information about the number of negative reaction fields. Thus, a table associating negative-positive information with the number of molecules or particles or concentration of the analyte is generated.

The table-generating section 404 may generate a table at any timing. For example, the table-generating section 404 may generate a table each time the analysis system 1 performs analysis, once in multiple times the analysis system 1 performs analysis, or in response to a user's manipulation of the analysis system 1. Alternatively, the table-generating section 404 may generate a table during regular maintenance of the analysis system 1 or before shipment or installation of the analysis system 1.

Concentration-Determining Section

The concentration-determining section 406 determines the number of molecules or particles or concentration of the analyte in the sample based on at least one piece of information selected from the group consisting of information about the number of positive reaction fields and information about the number of negative reaction fields and the table stored in the storage section 401. Although an example in which the concentration-determining section 406 determines the concentration of the analyte in the sample is described below, the same applies when the concentration-determining section 406 determines the number of molecules or particles of the analyte in the sample.

More specifically, the concentration-determining section 406 references the table used for concentration determination stored in the storage section 401 to acquire the number of molecules or particles of the analyte corresponding to the negative-positive information acquired by the analyte-information acquiring section 301. The concentration-determining section 406 then divides the acquired number of molecules or particles of the analyte by the total volume of the reaction fields subjected to detection by the analyte-information acquiring section 301 to calculate the concentration of the analyte in the reaction solution. The concentration-determining section 406 then multiplies the concentration of the analyte in the reaction solution by the dilution factor to calculate the concentration of the analyte in the sample. Alternatively, the concentration-determining section 406 may reference the table stored in the storage section 401 to directly acquire the concentration of the analyte in the reaction solution or the sample corresponding to the negative-positive information acquired by the analyte-information acquiring section 301.

The concentration-determining section 406 may determine the concentration of the analyte in the sample based on at least one of the proportion of the positive reaction fields and the proportion of the negative reaction fields and the table stored in the storage section 401. The use of such proportion information allows appropriate concentration determination even if the size distribution of the plurality of reaction fields obtained for table generation does not completely match the size distribution of the plurality of reaction fields subjected to detection by the analyte-information acquiring section 301.

As described above, the negative-positive information contained in the table generated by the table-generating section 404 is discrete. The negative-positive information contained in the table stored in the storage section 401 in this embodiment may be either discrete or continuous; in practice, the negative-positive information contained in the table is often discrete. If the negative-positive information contained in the table is discrete, it is possible that the table does not contain data matching the negative-positive information acquired by the analyte-information acquiring section 301. In such cases, the concentration-determining section 406 may interpolate the discrete negative-positive information contained in the table stored in the storage section 401 to determine the number of molecules or particles or concentration of the analyte in the sample. Interpolation may be accomplished in any manner. For example, interpolation may be accomplished by approximating values between two points by a straight line or curve based on two pieces of negative-positive information, contained in the table, that are closest to the negative-positive information acquired by the analyte-information acquiring section 301. Alternatively, the concentration may be determined based on negative-positive information, contained in the table, that is closest to the negative-positive information acquired by the analyte-information acquiring section 301.

Operation of Analysis System

An analysis method using the analysis system 1 according to this embodiment will next be described with reference to FIG. 3. FIG. 3 is a flowchart showing the steps of an analysis process performed by the analysis system 1 according to the first embodiment.

At S301, a sample for quantitative analysis of an analyte is prepared. Here, the sample is prepared, for example, by dilution and pretreatment of a specimen. The sample may be prepared within the analysis system 1 or may be prepared using a device outside the analysis system 1, for example, a commercially available specimen pretreatment device.

At S302, the reaction-field generating unit U1 splits a reaction solution containing the sample to generate a plurality of reaction fields independent of each other.

At S303, the reaction unit U2 allows a reaction to proceed in each of the plurality of reaction fields to make the analyte detectable.

At S304, the information processing unit U4 determines whether to generate a table. If a table is to be generated (Yes at S304), the process proceeds to S305. If no table is to be generated (No at S304), the process skips to S309. For example, if the information processing unit U4 does not include the table-generating section 404, the process may skip S304 and proceed to S309.

At S305, the detection unit U3 observes each of the plurality of reaction fields to detect the size of each of the plurality of reaction fields. Thus, the detection unit U3 acquires information about the size of each of the plurality of reaction fields.

At S306, the information processing unit U4 receives the information about the size of each of the reaction fields from the detection unit U3 and generates distribution data representing the size distribution of the reaction fields.

At S307, the information processing unit U4 generates a table based on the generated distribution data. In this embodiment, the table-generating section 404 generates, based on the distribution data, a table associating negative-positive information with the number of molecules or particles or concentration of the analyte. The details of the method for table generation by the information processing unit U4 are as described above.

At S308, the information processing unit U4 stores the generated table in the storage section 401.

At S309, the detection unit U3 observes each of the plurality of reaction fields to detect the analyte. Thus, the detection unit U3 acquires negative-positive information.

At S310, the information processing unit U4 determines the concentration of the analyte in the sample based on the negative-positive information acquired by the detection unit U3 and the table stored in the storage section 401. The details of the method for concentration determination by the concentration-determining section 406 of the information processing unit U4 are as described above.

Thus, in this embodiment, the concentration of the analyte is determined based on the negative-positive information acquired by the detection unit U3 using the table stored in the storage section 401. Because the table reflects information about the size distribution of the reaction fields, the reliability of analytical results can be improved even if there is variation in the size of the reaction fields. In addition, because in this embodiment the concentration is determined using a table in which information about the size distribution of the reaction fields is reflected in advance, the concentration-determining section 406 need not perform calculations by associating negative-positive information with size information for each reaction field when determining the concentration. Thus, according to this embodiment, the reliability of analytical results can be improved in a simpler manner than in the known art even if there is variation in the size of the reaction fields.

According to this embodiment, the information acquired by the analyte-information acquiring section 301 and the information acquired by the size-information acquiring section 302 need not be combined together. This is advantageous in that the size distribution and the negative-positive information can be handled separately.

According to this embodiment, if it can be assumed that reaction fields having the same size distribution or a size distribution with the same shape are generated each time reaction fields are generated, table generation may be omitted, and an existing table may be used (No at S304). This allows the steps of size detection and table generation to be omitted, thus speeding up the analysis. On the other hand, in this embodiment, the size distribution of the reaction fields may be acquired, and a table may be generated based on the size distribution. This allows the table to reliably reflect the size distribution of the reaction fields subjected to analysis, thus further improving the reliability of analytical results.

### Second Embodiment

An analysis system according to a second embodiment of the present invention will next be described with reference to the drawings. Components similar to those of the first embodiment are denoted by the same reference numerals as those of the first embodiment, and a detailed description thereof is omitted.

Configuration of Analysis System

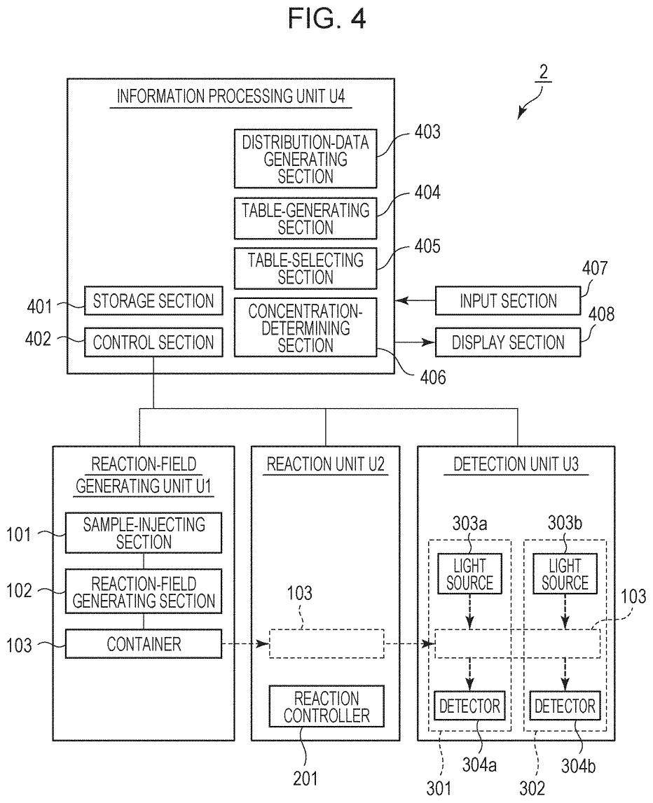

FIG. 4 schematically illustrates the configuration of the analysis system according to this embodiment. An analysis system 2 according to this embodiment includes a reaction-field generating unit U1, a reaction unit U2, a detection unit U3, and an information processing unit U4, as does the analysis system 1. The configurations of the reaction-field generating unit U1, the reaction unit U2, and the detection unit U3 of the analysis system 2 are similar to those of the analysis system 1.

Information Processing Unit

As shown in FIG. 4, the information processing unit U4 of the analysis system 2 according to this embodiment includes, as its function, a table-selecting section 405 in addition to the various sections of the information processing unit U4 of the analysis system 1.

Storage Section

The storage section 401 according to this embodiment is configured to store a plurality of tables. The plurality of tables stored in the storage section 401 correspond to different size distributions. Such a collection of tables may hereinafter be referred to as “library”.

Table-Selecting Section

The table-selecting section 405 selects the table used for concentration determination by the concentration-determining section 406 from the plurality of tables stored in the storage section 401 based on the distribution data generated by the distribution-data generating section 403. The table selected by the table-selecting section 405 is used for concentration determination by the concentration-determining section 406.

The table-selecting section 405 may determine whether there is a table suitable for the distribution data in the library stored in the storage section 401. If there is no table suitable for the distribution data, the table-generating section 404 may generate a table using the distribution data.

Operation of Analysis System

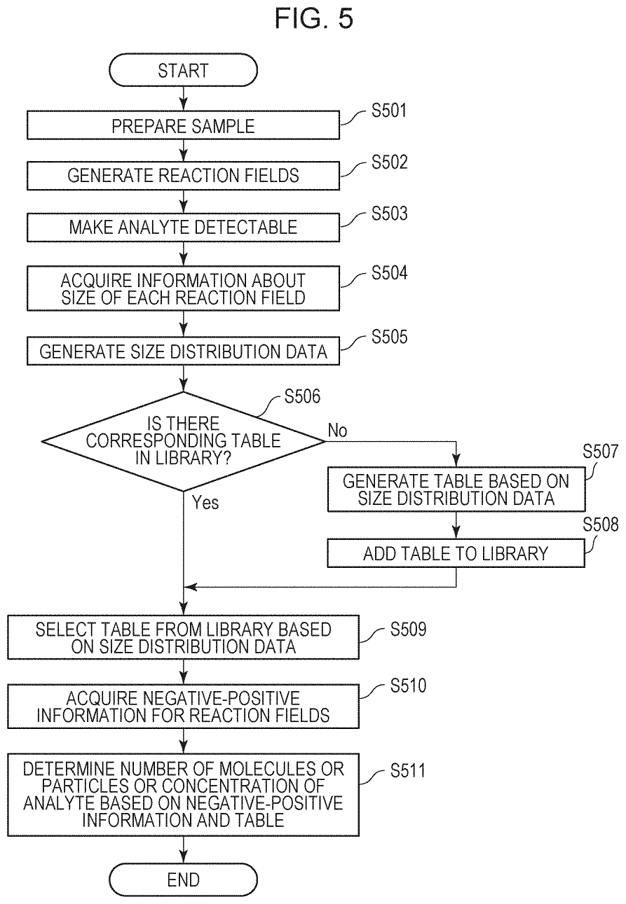

An analysis method using the analysis system 2 according to this embodiment will next be described with reference to FIG. 5. FIG. 5 is a flowchart showing the steps of an analysis process performed by the analysis system 2 according to the second embodiment.

S501 to S505 are similar to S301 to S303, S305, and S306; therefore, a description thereof is omitted.

At S506, the information processing unit U4 determines whether there is a table suitable for the distribution data generated by the distribution-data generating section 403 and representing the size distribution of the reaction fields in the library stored in the storage section 401. If there is a suitable table (Yes at S506), the process skips to S509. If there is no suitable table (No at S506), the process proceeds to S507. For example, if it is obvious that there is a suitable table in the library because the size distribution of the reaction fields is predicted to some degree in advance, the process may skip S506.

At S507, the information processing unit U4 generates a table based on the generated distribution data. In this embodiment, the table-generating section 404 generates, based on the distribution data, a table associating negative-positive information with the number of molecules or particles or concentration of the analyte. The details of the method for table generation by the information processing unit U4 are as described above.

At S508, the information processing unit U4 adds the generated table to the library and stores it in the storage section 401.

At S509, the table-selecting section 405 selects a table from the library stored in the storage section 401 based on the distribution data generated by the distribution-data generating section 403.

At S510, the detection unit U3 observes each of the plurality of reaction fields to detect the analyte. Thus, the detection unit U3 acquires negative-positive information for the plurality of reaction fields.

At S511, the information processing unit U4 determines the concentration of the analyte in the sample based on the negative-positive information acquired by the detection unit U3 and the table selected by the table-selecting section 405. The details of the method for concentration determination by the concentration-determining section 406 of the information processing unit U4 are similar to those of the first embodiment.

Thus, in this embodiment, the concentration of the analyte is determined based on the negative-positive information acquired by the detection unit U3, as in the first embodiment, using the table selected by the table-selecting section 405. The table reflects information about the size distribution of the reaction fields; therefore, as in the first embodiment, the reliability of analytical results can be improved in a simpler manner than in the known art even if there is variation in the size of the reaction fields.

In addition, according to this embodiment, once a table is acquired or generated, the table can be stored as a library and can be selected from the library and used when reaction fields having the same size distribution or a size distribution with the same shape are generated next. This allows the steps of size detection and table generation to be omitted, thus further speeding up the analysis.

### Third Embodiment

An analysis system according to a third embodiment of the present invention will next be described with reference to the drawings. A description of components similar to those of the foregoing embodiments is omitted.

Configuration of Analysis System

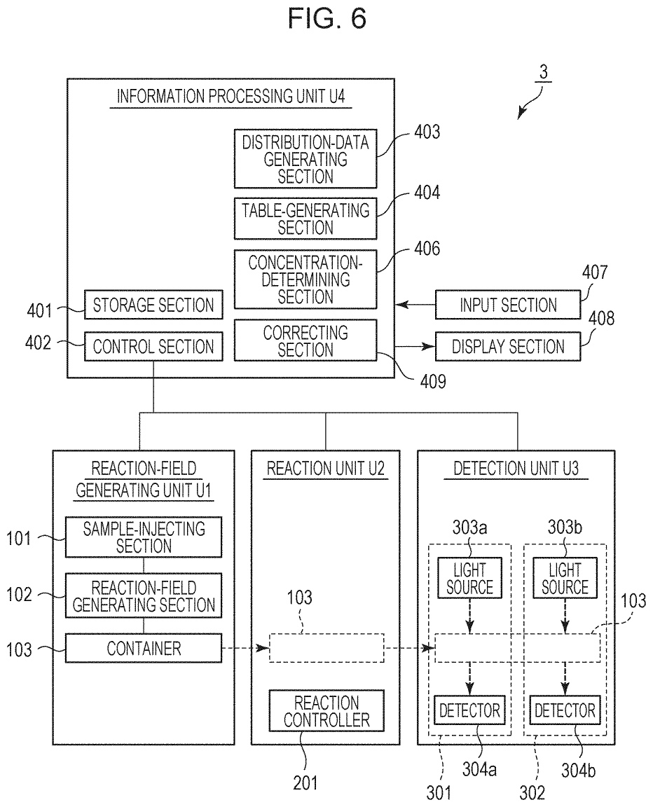

FIG. 6 schematically illustrates the configuration of the analysis system according to this embodiment. An analysis system 3 according to this embodiment includes a reaction-field generating unit U1, a reaction unit U2, a detection unit U3, and an information processing unit U4, as do the analysis systems 1 and 2. The configurations of the reaction-field generating unit U1, the reaction unit U2, and the detection unit U3 of the analysis system 3 are similar to those of the analysis systems 1 and 2.

Information Processing Unit

As shown in FIG. 6, the information processing unit U4 of the analysis system 3 according to this embodiment includes, as its function, a correcting section 409 in addition to the various sections of the information processing unit U4 of the analysis system 1.

Storage Section

The storage section 401 according to this embodiment is configured to store a table for correcting the number of molecules or particles or concentration of the analyte determined on the assumption that the size distribution of the reaction fields is monodisperse. The table may be a table associating information about the number of molecules or particles or concentration of the analyte determined on the assumption that the size distribution of the reaction fields is monodisperse with information about the number of molecules or particles or concentration of the analyte determined so as to reflect the size distribution of the reaction fields. The table may contain information about the difference between the information about the number of molecules or particles or concentration of the analyte determined on the assumption that the size distribution of the reaction fields is monodisperse and the information about the number of molecules or particles or concentration of the analyte determined so as to reflect the size distribution of the reaction fields.

The table stored in the storage section 401 may be a table theoretically generated based on information about the size of each of the plurality of reaction fields or may be a table experimentally acquired from standard samples with known analyte concentrations. The table stored in the storage section 401 may be a table theoretically generated based on information about the size of each of the plurality of reaction fields because such a table can be acquired without providing standard samples and can reflect the size distribution of the reaction fields.

Table-Generating Section

The table-generating section 404 according to this embodiment generates a table for concentration correction by the correcting section 409 based on the size information acquired by the size-information acquiring section 302. The table generated by the table-generating section 404 is stored in the storage section 401 and is used for concentration correction by the correcting section 409.

The table-generating section 404 may generate a table based on the size information acquired by the size-information acquiring section 302 by assuming the number of molecules or particles of the analyte and performing a simulation based on the Poisson model. A specific example of the method for table generation by the table-generating section 404 includes steps (1) to (6):

(1) The distribution data is received from the distribution-data generating section 403.

(2) The number of molecules or particles or concentration of the analyte present, before the generation of the plurality of reaction fields, in the liquid corresponding to the plurality of reaction fields subjected to detection for the acquisition of the distribution data (setting value 1) is assumed.

(3) The assumed analyte is sorted into each of the plurality of classes in the distribution data. Here, the analyte is sorted into each class in proportion to the proportion of the total volume of the reaction fields in the class to the total volume of all reaction fields.

(4) The number of reaction fields containing the analyte and the number of reaction fields containing no analyte in each of the plurality of classes in the distribution data are probabilistically calculated based on the Poisson model. The number of reaction fields containing the analyte is referred to as “number of positive reaction fields”, whereas the number of reaction fields containing no analyte is referred to as “number of negative reaction fields”. For example, the proportion of the reaction fields containing no analyte molecule or particle in each of the plurality of classes may be calculated based on the Poisson model, and the number of negative reaction fields and the number of positive reaction fields may be calculated based on the calculated proportion.

(5) The numbers of positive reaction fields calculated for the individual classes are added together to calculate the number of positive reaction fields in all reaction fields, and the numbers of negative reaction fields calculated for the individual classes are added together to calculate the number of negative reaction fields in all reaction fields.

(6) Using at least one of the calculated number of positive reaction fields and the calculated number of negative reaction fields, the number of molecules or particles or concentration of the analyte present in the liquid corresponding to the plurality of reaction fields before the generation of the plurality of reaction fields is calculated backwards on the assumption that the size distribution of the reaction fields is monodisperse. Thus, the number of molecules or particles or concentration of the analyte (setting value 1) is associated with the number of molecules or particles or concentration of the analyte determined from the negative-positive information probabilistically estimated based on the number of molecules or particles or concentration of the analyte and the size distribution of the reaction fields on the assumption that the size distribution of the reaction fields is monodisperse. In other words, in this way, the number of molecules or particles or concentration of the analyte determined on the assumption that the size distribution of the reaction fields is monodisperse is associated with the number of molecules or particles or concentration of the analyte determined so as to reflect the size distribution of the reaction fields (setting value 1).

Here, the number of molecules or particles or concentration of the analyte may be calculated by a known method of concentration calculation for digital analysis.

A situation in which it can be assumed that each reaction field contains either one or no analyte molecule or particle before the reaction in the reaction unit U2 will be described first. In this case, the number of positive reaction fields, x, can be assumed to be the number of molecules or particles of the analyte present in the volume Vs of the reaction solution subjected to analyte detection by the detection unit U3. Hence, the concentration λᵣ of the analyte in the reaction solution can be calculated from equation (1):

λᵣ=x/Vs equation (1)

The volume Vs subjected to analyte detection by the detection unit U3 can be calculated based on the information about the size of the reaction fields received from the detection unit U3.

Otherwise, for example, if it can be assumed that one reaction field can contain a plurality of analyte molecules or particles before the reaction in the reaction unit U2, the concentration of the analyte can be calculated by performing a correction based on the Poisson model. In this case, the concentration of the analyte is calculated by estimating the average number of molecules or particles, C, of the analyte present in each reaction field before the reaction in the reaction unit U2. Specifically, letting C be the average number of molecules or particles of the analyte present in one of the reaction fields subjected to analyte detection by the detection unit U3, the probability of one reaction field containing n analyte molecules or particles is expressed, from a formula based on the Poisson model, by equation (2):

$$P ( n , C ) = \frac{{c}^{n} \cdot {e}^{- C}}{n /} \qquad equation ( 2 )$$

Here, the probability of one reaction field containing no analyte molecule or particle is expressed by equation (2) where n=0, that is, by equation (3):

P(0,C)=e^{-c} equation (3)

If one reaction field contains at least one analyte molecule or particle before the reaction in the reaction unit U2, a signal can be detected from that reaction field. However, no information is available about the number of molecules or particles of the analyte present in that reaction field before the reaction. Accordingly, the number of molecules or particles of the analyte present in the reaction solution subjected to detection is estimated using equation (3) based on the proportion of the number of negative reaction fields to the total number of reaction fields subjected to detection by the detection unit U3.

Specifically, the proportion F₀ of reaction fields in which no signal has been detected is calculated from the number of positive reaction fields or negative reaction fields and the total number of reaction fields subjected to detection. The average number of molecules or particles, C, of the analyte present in one of the reaction fields subjected to detection before the reaction in the reaction unit U2 is estimated from equation (4):

C=−ln(F₀) equation (4)

Here, letting v be the average volume of the reaction fields subjected to analyte detection by the detection unit U3, the concentration λ_{c} of the analyte in the reaction solution can be calculated from equation (5):

λ_{c}=C/v equation (5)

The average volume v subjected to analyte detection by the detection unit U3 can be calculated based on the information about the size of the reaction fields received from the detection unit U3.

Alternatively, the concentration λ_{c} of the analyte in the reaction solution may be calculated based on the total number of molecules or particles of the analyte obtained by multiplying the average number of molecules or particles, C, of the analyte by the number of reaction fields, and the total volume of the reaction fields obtained by multiplying the average volume v of the reaction fields by the number of reaction fields.

The thus-calculated concentration of the analyte in the reaction solution can be converted to the concentration of the analyte in the specimen or the sample using the dilution factor used in the preparation of the reaction solution from the specimen or the sample.

(7) Steps (2) to (6) are repeated with varying setting values. Thus, each of n setting values (setting values 1 to n) is associated with the number of molecules or particles or concentration of the analyte calculated backwards from the negative-positive information obtained based on the size distribution of the reaction fields and the Poisson model on the assumption that the size distribution of the reaction fields is monodisperse. Thus, a table is generated that associates information about the number of molecules or particles or concentration of the analyte determined on the assumption that the size distribution of the reaction fields is monodisperse with information about the number of molecules or particles or concentration of the analyte determined so as to reflect the size distribution of the reaction fields.

The table-generating section 404 may generate a table at any timing. For example, the table-generating section 404 may generate a table each time the analysis system 3 performs analysis, once in multiple times the analysis system 3 performs analysis, or in response to a user's manipulation of the analysis system 3. Alternatively, the table-generating section 404 may generate a table during regular maintenance of the analysis system 3 or before shipment or installation of the analysis system 3.

Concentration-Determining Section

The concentration-determining section 406 according to this embodiment determines the number of molecules or particles or concentration of the analyte in the sample based on the negative-positive information acquired by the analyte-information acquiring section 301 on the assumption that the size distribution of the reaction fields is monodisperse.

Correcting Section

The correcting section 409 corrects the number of molecules or particles or concentration of the analyte in the sample determined by the concentration-determining section 406 using the table stored in the storage section 401.

Operation of Analysis System

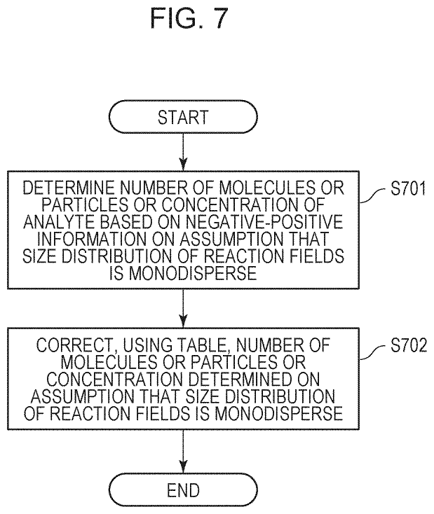

An analysis method using the analysis system 3 according to this embodiment will next be described with reference to FIGS. 3 and 7. FIG. 7 is a flowchart showing some of the steps of analysis processes performed by the analysis systems 3 and 4 according to the third and fourth embodiments.

S301 to S306 are similar to those of the first embodiment.

At S307, the information processing unit U4 generates a table based on the generated distribution data. In this embodiment, the table-generating section 404 generates, based on the distribution data, a table for correcting the number of molecules or particles or concentration of the analyte determined on the assumption that the size distribution of the reaction fields is monodisperse. The details of the method for table generation by the information processing unit U4 are as described above.

S308 and S309 are similar to those of the first embodiment.

At S310, the information processing unit U4 determines the concentration of the analyte in the sample based on the negative-positive information acquired by the detection unit U3 and the table stored in the storage section 401. Here, in this embodiment, the concentration is determined by the steps shown in FIG. 7.

At S701, the information processing unit U4 determines the number of molecules or particles or concentration of the analyte based on the negative-positive information acquired by the detection unit U3 on the assumption that the size distribution of the reaction fields is monodisperse.

At S702, the information processing unit U4 corrects the number of molecules or particles or concentration determined at S701 using the table stored in the storage section 401.

Thus, in this embodiment, the concentration of the analyte in the sample is acquired by correcting the concentration determined with the size distribution ignored, that is, on the assumption that the size distribution is monodisperse, using the table stored in the storage section 401. In this embodiment, as in the first embodiment, the table reflects information about the size distribution of the reaction fields; therefore, the reliability of analytical results can be improved in a simpler manner than in the known art even if there is variation in the size of the reaction fields. According to this embodiment, other advantages described in the first embodiment can also be achieved.

### Fourth Embodiment

An analysis system according to a fourth embodiment of the present invention will next be described with reference to the drawings. A description of components similar to those of the foregoing embodiments is omitted.

Configuration of Analysis System

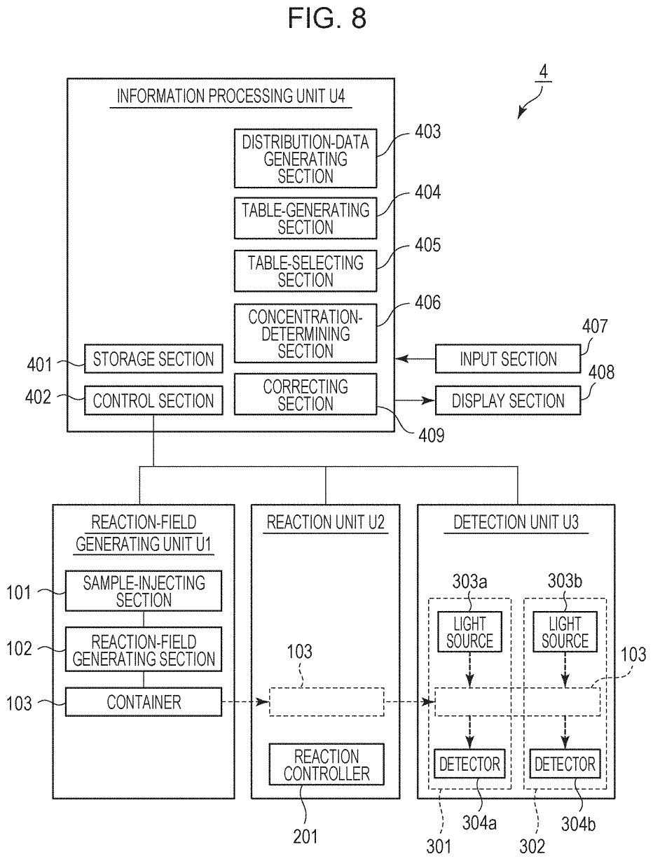

FIG. 8 schematically illustrates the configuration of the analysis system according to this embodiment. An analysis system 4 according to this embodiment includes a reaction-field generating unit U1, a reaction unit U2, a detection unit U3, and an information processing unit U4, as do the analysis systems 1 to 3. The configurations of the reaction-field generating unit U1, the reaction unit U2, and the detection unit U3 of the analysis system 4 are similar to those of the analysis systems 1 to 3.

Information Processing Unit

As shown in FIG. 8, the information processing unit U4 of the analysis system 4 according to this embodiment includes, as its function, a table-selecting section 405 in addition to the various sections of the information processing unit U4 of the analysis system 3.

Storage Section

The storage section 401 according to this embodiment is configured to store a plurality of tables. The plurality of tables stored in the storage section 401 correspond to different size distributions. Such a collection of tables may hereinafter be referred to as “library”.

Table-Selecting Section

The table-selecting section 405 selects the table used for concentration correction by the correcting section 409 from the plurality of tables stored in the storage section 401 based on the distribution data generated by the distribution-data generating section 403. The table selected by the table-selecting section 405 is used for concentration correction by the correcting section 409.

The table-selecting section 405 may determine whether there is a table suitable for the distribution data in the library stored in the storage section 401. If there is no table suitable for the distribution data, the table-generating section 404 may generate a table using the distribution data.

Operation of Analysis System

An analysis method using the analysis system 4 according to this embodiment will next be described with reference to FIGS. 5 and 7. FIG. 7 is a flowchart showing some of the steps of the analysis processes performed by the analysis systems 3 and 4 according to the third and fourth embodiments.

S501 to S506 are similar to those of the second embodiment.

At S507, the information processing unit U4 generates a table based on the generated distribution data. In this embodiment, the table-generating section 404 generates, based on the distribution data, a table for correcting the number of molecules or particles or concentration of the analyte determined on the assumption that the size distribution of the reaction fields is monodisperse. The details of the method for table generation by the information processing unit U4 are similar to those of the third embodiment.

S508 to S510 are similar to those of the second embodiment.

At S511, the information processing unit U4 determines the concentration of the analyte in the sample based on the negative-positive information acquired by the detection unit U3 and the table selected by the table-selecting section 405. Here, in this embodiment, as in the third embodiment, the concentration is determined by the steps shown in FIG. 7.

S701 and S702 are similar to those of the third embodiment.

Thus, in this embodiment, the concentration of the analyte in the sample is acquired by correcting the concentration determined with the size distribution ignored, that is, on the assumption that the size distribution is monodisperse, using the table selected by the table-selecting section 405. In this embodiment, as in the second embodiment, the table reflects information about the size distribution of the reaction fields; therefore, the reliability of analytical results can be improved even if there is variation in the size of the reaction fields. According to this embodiment, other advantages described in the second embodiment can also be achieved.

### Other Embodiments

Whereas the analysis systems 1 to 4 according to the embodiments of the present invention have been described, the invention is not limited thereto, but can also be embodied as an analysis system composed of some of the various sections forming the analysis systems 1 to 4.

Whereas examples in which the number of molecules or particles or concentration of the analyte determined on the assumption that the size distribution of the reaction fields is monodisperse is corrected using a table have been described in the third and fourth embodiments, the present invention is not limited thereto. For example, the negative-positive information may be corrected using a table, and the corrected negative-positive information may be used to determine the number of molecules or particles or concentration of the analyte on the assumption that the size distribution of the reaction fields is monodisperse. That is, in the third and fourth embodiments, the storage section 401 may store a table for correcting the negative-positive information, and the correcting section 409 may correct the negative-positive information based on the table stored in the storage section 401. The concentration-determining section 406 may then determine the number of molecules or particles or concentration of the analyte based on the negative-positive information corrected by the correcting section 409.

The present invention can also be embodied as a program configured to be supplied to a system or device via a network or a computer-readable storage medium and to be read and executed by one or more processors in a computer of the system or device to implement one or more functions of the foregoing embodiments. The invention can also be embodied as a circuit (e.g., an ASIC) configured to implement one or more functions.

### EXAMPLES

The present invention will now be described in more detail with reference to the following examples, although these examples are not intended to limit the invention.

### Preparation Example 1

Generation of Emulsion 1

Two microliters of a 10-fold dilution of QuickPrimer Control DNA 5 (5 ng/μL, product code MR405, available from Takara Bio Inc.), 4 μL of QuickPrimer Escherichia/Shigella group (16S rDNA) (2.0 μM each of forward and reverse primers, product code MR201, available from Takara Bio Inc.), 10 μL of SYBR Premix Ex Taq (Tli RNaseH Plus) (product code RR420, available from Takara Bio Inc.), and 4 μL of sterile distilled water were prepared and mixed together.

This mixture was subjected to PCR by thermal cycling under the following thermal cycling conditions to obtain amplicons of Escherichia coli 16S rDNA. Agarose gel electrophoresis showed that 413-bp amplification products were obtained. The solution was diluted to an amplicon concentration of about 5×10⁴ copies/μL to obtain Template 1.

Thermal Cycling Conditions

1) Initial denaturation (95° C. for 2 minutes): 1 cycle

2) PCR (95° C. for 20 seconds, 55° C. for 20 seconds, and 74° C. for 20 seconds): 35 cycles

3) Retention (4° C.): 1 cycle

To 10 μL of a commercially available PCR reagent for intercalator methods (ddPCR EvaGreen Supermix, available from Bio-Rad Laboratories, Inc.), 2 μL of Template 1 above, 1 μL of QuickPrimer Escherichia/Shigella group (16S rDNA) (2.0 μM each of forward and reverse primers, product code MR201, available from Takara Bio Inc.), and 7 μL of sterile distilled water were added and mixed to obtain a dispersed phase.

To the dispersed phase, 50 μL of a commercially available oil for digital PCR (Droplet Generator Oil for Evagreen, available from Bio-Rad Laboratories, Inc.) was added as a continuous phase. This mixture was agitated on a vortex mixer to obtain Emulsion 1 as a water-in-oil emulsion.

PCR Using Emulsion

Emulsion 1 thus obtained was subjected to PCR by thermal cycling under the following thermal cycling conditions.

Thermal Cycling Conditions

1) Enzyme activation (95° C. for 5 minutes): 1 cycle

2) PCR (95° C. for 30 seconds and 55° C. for 1 minute): 50 cycles

3) Signal stabilization (4° C. for 5 minutes and 90° C. for 5 minutes): 1 cycle

4) Retention (4° C.): 1 cycle

Droplet Measurement after Thermal Cycling

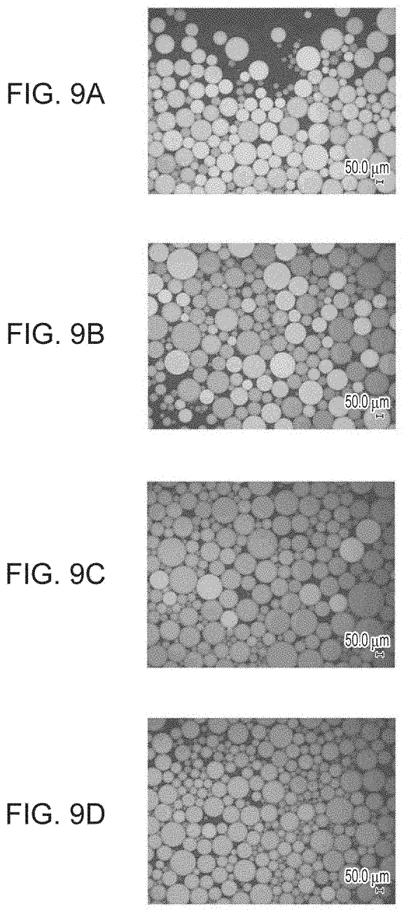

Onto a glass plate for sediment examination (MUR-300, available from Matsunami Glass Ind., Ltd.) was placed 20 μL of Emulsion 1 after thermal cycling. The emulsion was observed under a fluorescence microscope (BZ-8000, available from Keyence Corporation). Observation was performed in five fields of view. For each field of view, a visible light image and a fluorescence image (excitation wavelength: 480/30 nm, absorption wavelength: 510 nm) of the same field of view were captured with a built-in camera (image capture device: 1,500,000-pixel CCD image sensor). An example of a captured image is shown in FIG. 9A.

The resulting visible light images were analyzed using image processing software to measure the diameter of each droplet. The measurement resolution was 10 μm. Droplets with diameters of 10 μm or less were excluded from the measurement. In addition, the resulting fluorescence images were visually inspected for the presence or absence of fluorescence enhancement due to gene amplification for each droplet to determine whether the analyte was detected. The visible light images and the fluorescence images were superimposed on top of each other to generate data associating information about the size of each droplet with information about whether the analyte was detected.

The resulting data was used to generate frequency distribution data by dividing the droplet size into a plurality of classes. Specifically, droplet diameters of from 20 μm to less than 30 μm were defined as one class, and the subsequent classes were similarly defined at intervals of 10 μm, which is the measurement resolution, thereby dividing the droplet diameter into 18 classes. The number of droplets, the number of positive droplets, and the number of negative droplets were then counted for each class. The results are summarized in Table 1.

TABLE 1

| | | Emulsion 1 | | | Emulsion 2 | | | Emulsion 3 | | | Emulsion 4 | | |
|---|---|---|---|---|---|---|---|---|---|---|---|---|---|
| Droplet | | Number of droplets | | | Number of droplets | | | Number of droplets | | | Number of droplets | | |
| diameter | | (droplets) | | | (droplets) | | | (droplets) | | | (droplets) | | |
| (μm) | Class | Total | Positive | Negative | Total | Positive | Negative | Total | Positive | Negative | Total | Positive | Negative |
| 20 | Class 1 | 47 | 4 | 43 | 99 | 1 | 98 | 275 | 0 | 275 | 165 | 0 | 165 |
| 30 | Class 2 | 35 | 6 | 29 | 85 | 1 | 84 | 252 | 0 | 252 | 136 | 0 | 136 |
| 40 | Class 3 | 45 | 7 | 38 | 69 | 1 | 68 | 175 | 1 | 174 | 114 | 0 | 114 |
| 50 | Class 4 | 60 | 24 | 36 | 41 | 0 | 41 | 132 | 0 | 132 | 81 | 0 | 81 |
| 60 | Class 5 | 35 | 20 | 15 | 30 | 1 | 29 | 98 | 2 | 96 | 71 | 0 | 71 |
| 70 | Class 6 | 38 | 24 | 14 | 42 | 3 | 39 | 79 | 1 | 78 | 55 | 0 | 55 |
| 80 | Class 7 | 34 | 32 | 2 | 23 | 4 | 19 | 77 | 0 | 77 | 49 | 1 | 48 |
| 90 | Class 8 | 32 | 26 | 6 | 35 | 5 | 30 | 66 | 0 | 66 | 44 | 1 | 43 |
| 100 | Class 9 | 41 | 39 | 2 | 50 | 12 | 38 | 78 | 1 | 77 | 47 | 0 | 47 |
| 110 | Class 10 | 46 | 46 | 0 | 37 | 7 | 30 | 68 | 2 | 66 | 51 | 1 | 50 |
| 120 | Class 11 | 33 | 31 | 2 | 32 | 12 | 20 | 64 | 1 | 63 | 41 | 0 | 41 |
| 130 | Class 12 | 34 | 32 | 2 | 38 | 12 | 26 | 60 | 4 | 56 | 27 | 0 | 27 |
| 140 | Class 13 | 17 | 16 | 1 | 25 | 7 | 18 | 39 | 0 | 39 | 26 | 0 | 26 |
| 150 | Class 14 | 9 | 9 | 0 | 29 | 9 | 20 | 38 | 2 | 36 | 27 | 0 | 27 |
| 160 | Class 15 | 10 | 10 | 0 | 14 | 8 | 6 | 32 | 4 | 28 | 13 | 0 | 13 |
| 170 | Class 16 | 0 | 0 | 0 | 6 | 2 | 4 | 25 | 2 | 23 | 16 | 1 | 15 |
| 180 | Class 17 | 2 | 2 | 0 | 3 | 3 | 0 | 25 | 4 | 21 | 11 | 1 | 10 |
| 190 | Class 18 | 0 | 0 | 0 | 4 | 1 | 3 | 13 | 1 | 12 | 9 | 0 | 9 |

In the table, “droplet diameter” shows the average diameter of droplets, “total” shows the total number of droplets, “positive” shows the number of droplets with fluorescence enhancement (positive droplets), and “negative” shows the number of droplets without fluorescence enhancement (negative droplets) (the same applies hereinafter).

### Preparation Example 2

In Preparation Example 1, Template 1 was diluted 10-fold to obtain Template 2. Thus, Template 2 had an amplicon concentration of about 5×10³ copies/μL. Emulsion 2 was generated as in Preparation Example 1 except that Template 2 was used instead of Template 1 to form a dispersed phase.

Emulsion 2 was subjected to PCR by thermal cycling as in Preparation Example 1. The droplets after thermal cycling were subjected to measurement as in Preparation Example 1. An example of a captured image is shown in FIG. 9B. The measurement results are summarized in Table 1.

### Preparation Example 3

In Preparation Example 1, Template 1 was diluted 100-fold to obtain Template 3. Thus, Template 3 had an amplicon concentration of about 5×10² copies/μL. Emulsion 3 was generated as in Preparation Example 1 except that Template 3 was used instead of Template 1 to form a dispersed phase.

Emulsion 3 was subjected to PCR by thermal cycling as in Preparation Example 1. The droplets after thermal cycling were subjected to measurement as in Preparation Example 1. An example of a captured image is shown in FIG. 9C. The measurement results are summarized in Table 1.

### Preparation Example 4

In Preparation Example 1, Template 1 was diluted 1,000-fold to obtain Template 4. Thus, Template 4 had an amplicon concentration of about 5×10 copies/μL. Emulsion 4 was generated as in Preparation Example 1 except that Template 4 was used instead of Template 1 to form a dispersed phase.

Emulsion 4 was subjected to PCR by thermal cycling as in Preparation Example 1. The droplets after thermal cycling were subjected to measurement as in Preparation Example 1. An example of a captured image is shown in FIG. 9D. The measurement results are summarized in Table 1.

### Preparation Example 5

In Preparation Example 1, PCR was performed as in Preparation Example 1 except that human 3-actin (Human ACTB Endogenous Control, Thermo Fisher Scientific Inc.) was used instead of 16S rDNA (QuickPrimer Escherichia/Shigella group). The solution was diluted to a human 3-actin amplicon concentration of about 5×10⁴ copies/μL to obtain Template 5. Emulsion 5 was generated as in Preparation Example 1 except that Template 5 was used instead of Template 1 to form a dispersed phase.

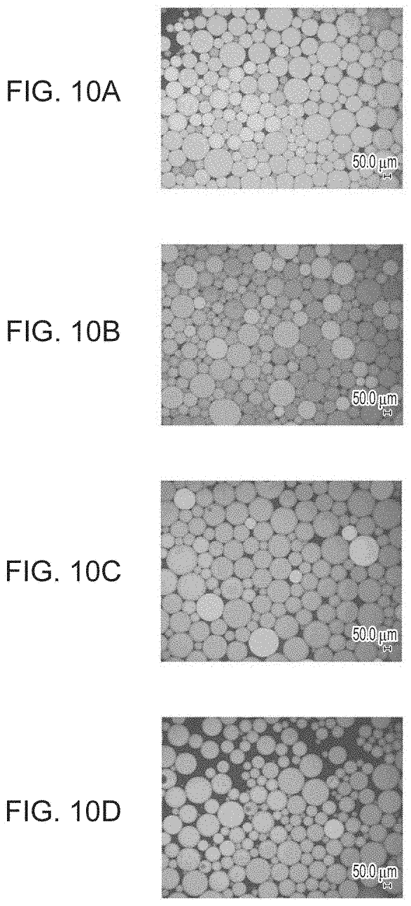

Emulsion 5 was subjected to PCR by thermal cycling as in Preparation Example 1. The droplets after thermal cycling were subjected to measurement as in Preparation Example 1. An example of a captured image is shown in FIG. 10A. The measurement results are summarized in Table 2.

TABLE 2

| | | Emulsion 5 | | | Emulsion 6 | | | Emulsion 7 | | | Emulsion 8 | | |
|---|---|---|---|---|---|---|---|---|---|---|---|---|---|
| Droplet | | Number of droplets | | | Number of droplets | | | Number of droplets | | | Number of droplets | | |
| diameter | | (droplets) | | | (droplets) | | | (droplets) | | | (droplets) | | |
| (μm) | Class | Total | Positive | Negative | Total | Positive | Negative | Total | Positive | Negative | Total | Positive | Negative |
| 20 | Class 1 | 172 | 8 | 164 | 200 | 3 | 197 | 167 | 0 | 167 | 51 | 0 | 51 |
| 30 | Class 2 | 96 | 7 | 89 | 125 | 4 | 121 | 130 | 0 | 130 | 40 | 0 | 40 |
| 40 | Class 3 | 76 | 20 | 56 | 71 | 4 | 67 | 93 | 0 | 93 | 86 | 0 | 86 |
| 50 | Class 4 | 44 | 17 | 27 | 76 | 5 | 71 | 74 | 0 | 74 | 81 | 0 | 81 |
| 60 | Class 5 | 51 | 28 | 23 | 70 | 9 | 61 | 43 | 0 | 43 | 82 | 0 | 82 |
| 70 | Class 6 | 56 | 44 | 12 | 56 | 5 | 51 | 35 | 0 | 35 | 81 | 0 | 81 |
| 80 | Class 7 | 53 | 45 | 8 | 52 | 14 | 38 | 34 | 1 | 33 | 48 | 0 | 48 |
| 90 | Class 8 | 43 | 41 | 2 | 56 | 12 | 44 | 35 | 2 | 33 | 44 | 0 | 44 |
| 100 | Class 9 | 39 | 39 | 0 | 44 | 7 | 37 | 29 | 1 | 28 | 48 | 0 | 48 |
| 110 | Class 10 | 38 | 37 | 1 | 42 | 11 | 31 | 26 | 0 | 26 | 42 | 1 | 41 |
| 120 | Class 11 | 37 | 36 | 1 | 35 | 8 | 27 | 25 | 1 | 24 | 40 | 1 | 39 |
| 130 | Class 12 | 38 | 37 | 1 | 37 | 18 | 19 | 28 | 0 | 28 | 49 | 2 | 47 |
| 140 | Class 13 | 22 | 22 | 0 | 35 | 9 | 26 | 25 | 0 | 25 | 34 | 0 | 34 |
| 150 | Class 14 | 15 | 15 | 0 | 23 | 7 | 16 | 23 | 2 | 21 | 23 | 1 | 22 |
| 160 | Class 15 | 29 | 29 | 0 | 26 | 12 | 14 | 17 | 2 | 15 | 18 | 0 | 18 |
| 170 | Class 16 | 16 | 16 | 0 | 16 | 8 | 8 | 15 | 1 | 14 | 20 | 0 | 20 |
| 180 | Class 17 | 10 | 10 | 0 | 11 | 7 | 4 | 10 | 0 | 10 | 16 | 2 | 14 |
| 190 | Class 18 | 6 | 6 | 0 | 8 | 5 | 3 | 7 | 0 | 7 | 7 | 0 | 7 |

### Preparation Example 6

In Preparation Example 5, Template 5 was diluted 10-fold to obtain Template 6. Thus, Template 6 had an amplicon concentration of about 5×10³ copies/μL. Emulsion 6 was generated as in Preparation Example 5 except that Template 6 was used instead of Template 5 to form a dispersed phase.

Emulsion 6 was subjected to PCR by thermal cycling as in Preparation Example 5. The droplets after thermal cycling were subjected to measurement as in Preparation Example 5. An example of a captured image is shown in FIG. 10B. The measurement results are summarized in Table 2.

### Preparation Example 7

In Preparation Example 5, Template 5 was diluted 100-fold to obtain Template 7. Thus, Template 7 had an amplicon concentration of about 5×10² copies/μL. Emulsion 7 was generated as in Preparation Example 5 except that Template 7 was used instead of Template 5 to form a dispersed phase.

Emulsion 7 was subjected to PCR by thermal cycling as in Preparation Example 5. The droplets after thermal cycling were subjected to measurement as in Preparation Example 5. An example of a captured image is shown in FIG. 10C. The measurement results are summarized in Table 2.

### Preparation Example 8

In Preparation Example 5, Template 5 was diluted 1,000-fold to obtain Template 8. Thus, Template 8 had an amplicon concentration of about 5×10 copies/μL. Emulsion 8 was generated as in Preparation Example 5 except that Template 8 was used instead of Template 5 to form a dispersed phase.

Emulsion 8 was subjected to PCR by thermal cycling as in Preparation Example 5. The droplets after thermal cycling were subjected to measurement as in Preparation Example 5. An example of a captured image is shown in FIG. 10D. The measurement results are summarized in Table 2.

### Comparative Example 1

For each of Emulsions 1 to 4 of Preparation Examples 1 to 4, the concentration of the analyte (target nucleic acid) in the sample was calculated by a method commonly used in digital PCR using droplets. Specifically, the sum of the total numbers of droplets in all classes and the sum of the numbers of negative droplets in all classes were used to calculate the proportion of the negative droplets. The average number of copies, C, of the analyte present in one reaction field was calculated using equation (4). The average number of copies, C, and the sum of the total numbers of droplets in all classes were then multiplied together to calculate the total number of copies of the analyte present in all droplets subjected to detection. Thereafter, the volume of the droplets present in each class was calculated from the total number of droplets in each class and the diameter of the droplets in that class, and the total volume of the droplets subjected to detection was calculated. The total number of copies of the analyte present in all droplets subjected to detection was then divided by the total volume of all droplets subjected to detection to calculate the concentration in the reaction solution. The calculated concentration was converted to the concentration of the analyte in the sample by multiplication by a dilution factor of 10. The results are shown in Table 3.

TABLE 3

| | | Total number of copies of | |
|---|---|---|---|
| | Relative | analyte present in all droplets | Concentration of |
| | dilution | subjected to detection | analyte in sample |
| Emulsion | factor | (copies) | (copies/μL) |
| Emulsion 1 | 1 | 520 | 22,700 |
| Emulsion 2 | 10 | 96 | 3,020 |
| Emulsion 3 | 100 | 25 | 364 |
| Emulsion 4 | 1,000 | 5 | 123 |

### Example 1

For each of Emulsions 1 to 4, a table associating the numbers of positive droplets and negative droplets with the number of copies of the analyte was generated based on the size distribution of the droplets in the emulsion. The concentration of the analyte was calculated based on the table and the negative-positive information.

Table Generation

The size distributions of the droplets in Emulsions 1 to 4 after thermal cycling were as shown in Table 1. For each of Emulsions 1 to 4, a table (calibration curve) associating the numbers of positive droplets and negative droplets with the number of copies of the analyte (the number of copies of the target nucleic acid) was generated based on the size distribution by the procedure described in the first embodiment.

A specific table generation procedure will now be described using Emulsion 1 as an example. First, the total number of copies of the analyte present in the plurality of droplets having the size distribution shown in Table 1 before emulsion formation (setting value) was assumed to be 100,000 copies. The assumed 100,000 copies of the analyte were then sorted into each of the plurality of classes shown in Table 1. The analyte was sorted into each class in proportion to the proportion of the total volume of the droplets present in the class to the total volume of all droplets.

The proportion of droplets containing no copy of the analyte in each of the plurality of classes was then probabilistically calculated based on the Poisson model. The resulting proportion was then subtracted from 1 to calculate the proportion of droplets containing one or more copies of the analyte. All droplets containing no copy of the analyte were regarded as negative droplets, whereas all droplets containing one or more copies of the analyte were regarded as positive droplets. The resulting proportions and the total number of droplets present in the class were multiplied together to calculate the numbers of negative droplets and positive droplets.

The total number of copies of the analyte present before emulsion formation (setting value) was also assumed to be 30,000 copies, 10,000 copies, 3,000 copies, 1,000 copies, 300 copies, 100 copies, 30 copies, 10 copies, and 3 copies, and calculations were similarly performed to calculate the numbers of negative droplets and positive droplets. The calculation results are summarized in Table 4.

TABLE 4

| | | | Number of copies of analyte (copies) | | | | | | | | | |
|---|---|---|---|---|---|---|---|---|---|---|---|---|
| Droplet | | | 100,000 | | 30,000 | | 10,000 | | 3,000 | | 1,000 | |
| diameter | | | Positive | Negative | Positive | Negative | Positive | Negative | Positive | Negative | Positive | Negative |
| (μm) | Class | Total | (droplets) | (droplets) | (droplets) | (droplets) | (droplets) | (droplets) | (droplets) | (droplets) | (droplets) | (droplets) |
| 20 | Class 1 | 47 | 39 | 8 | 20 | 27 | 8 | 39 | 3 | 44 | 1 | 46 |
| 30 | Class 2 | 35 | 35 | 0 | 30 | 5 | 16 | 19 | 6 | 29 | 2 | 33 |
| 40 | Class 3 | 45 | 45 | 0 | 44 | 1 | 35 | 10 | 16 | 29 | 6 | 39 |
| 50 | Class 4 | 60 | 60 | 0 | 60 | 0 | 57 | 3 | 35 | 25 | 15 | 45 |
| 60 | Class 5 | 35 | 35 | 0 | 35 | 0 | 35 | 0 | 27 | 8 | 14 | 21 |
| 70 | Class 6 | 38 | 38 | 0 | 38 | 0 | 38 | 0 | 34 | 4 | 21 | 17 |
| 80 | Class 7 | 34 | 34 | 0 | 34 | 0 | 34 | 0 | 33 | 1 | 23 | 11 |
| 90 | Class 8 | 32 | 32 | 0 | 32 | 0 | 32 | 0 | 32 | 0 | 26 | 6 |
| 100 | Class 9 | 41 | 41 | 0 | 41 | 0 | 41 | 0 | 41 | 0 | 37 | 4 |
| 110 | Class 10 | 46 | 46 | 0 | 46 | 0 | 46 | 0 | 46 | 0 | 44 | 2 |
| 120 | Class 11 | 33 | 33 | 0 | 33 | 0 | 33 | 0 | 33 | 0 | 32 | 1 |
| 130 | Class 12 | 34 | 34 | 0 | 34 | 0 | 34 | 0 | 34 | 0 | 34 | 0 |
| 140 | Class 13 | 17 | 17 | 0 | 17 | 0 | 17 | 0 | 17 | 0 | 17 | 0 |
| 150 | Class 14 | 9 | 9 | 0 | 9 | 0 | 9 | 0 | 9 | 0 | 9 | 0 |
| 160 | Class 15 | 10 | 10 | 0 | 10 | 0 | 10 | 0 | 10 | 0 | 10 | 0 |
| 170 | Class 16 | 0 | 0 | 0 | 0 | 0 | 0 | 0 | 0 | 0 | 0 | 0 |
| 180 | Class 17 | 2 | 2 | 0 | 2 | 0 | 2 | 0 | 2 | 0 | 2 | 0 |
| 190 | Class 18 | 0 | 0 | 0 | 0 | 0 | 0 | 0 | 0 | 0 | 0 | 0 |
| | | | Number of copies of analyte (copies) | | | | | | | | | |
| Droplet | | | 300 | | 100 | | 30 | | 10 | | 3 | |
| diameter | | | Positive | Negative | Positive | Negative | Positive | Negative | Positive | Negative | Positive | Negative |
| (μm) | Class | Total | (droplets) | (droplets) | (droplets) | (droplets) | (droplets) | (droplets) | (droplets) | (droplets) | (droplets) | (droplets) |
| 20 | Class 1 | 47 | 0 | 47 | 0 | 47 | 0 | 47 | 0 | 47 | 0 | 47 |
| 30 | Class 2 | 35 | 1 | 34 | 0 | 35 | 0 | 35 | 0 | 35 | 0 | 35 |
| 40 | Class 3 | 45 | 2 | 43 | 1 | 44 | 0 | 45 | 0 | 45 | 0 | 45 |
| 50 | Class 4 | 60 | 5 | 55 | 2 | 58 | 1 | 59 | 0 | 60 | 0 | 60 |
| 60 | Class 5 | 35 | 5 | 30 | 2 | 33 | 1 | 34 | 0 | 35 | 0 | 35 |
| 70 | Class 6 | 38 | 8 | 30 | 3 | 35 | 1 | 37 | 0 | 38 | 0 | 38 |
| 80 | Class 7 | 34 | 10 | 24 | 4 | 30 | 1 | 33 | 0 | 34 | 0 | 34 |
| 90 | Class 8 | 32 | 13 | 19 | 5 | 27 | 2 | 30 | 1 | 31 | 0 | 32 |
| 100 | Class 9 | 41 | 20 | 21 | 8 | 33 | 3 | 38 | 1 | 40 | 0 | 41 |
| 110 | Class 10 | 46 | 28 | 18 | 12 | 34 | 4 | 42 | 1 | 45 | 0 | 46 |
| 120 | Class 11 | 33 | 23 | 10 | 11 | 22 | 4 | 29 | 1 | 32 | 0 | 33 |
| 130 | Class 12 | 34 | 26 | 8 | 13 | 21 | 5 | 29 | 2 | 32 | 1 | 33 |
| 140 | Class 13 | 17 | 14 | 3 | 8 | 9 | 3 | 14 | 1 | 16 | 0 | 17 |
| 150 | Class 14 | 9 | 8 | 1 | 5 | 4 | 2 | 7 | 1 | 8 | 0 | 9 |
| 160 | Class 15 | 10 | 9 | 1 | 6 | 4 | 2 | 8 | 1 | 9 | 0 | 10 |
| 170 | Class 16 | 0 | 0 | 0 | 0 | 0 | 0 | 0 | 0 | 0 | 0 | 0 |
| 180 | Class 17 | 2 | 2 | 0 | 1 | 1 | 1 | 1 | 0 | 2 | 0 | 2 |
| 190 | Class 18 | 0 | 0 | 0 | 0 | 0 | 0 | 0 | 0 | 0 | 0 | 0 |

Based on Table 4, the numbers of positive droplets in all classes and the numbers of negative droplets in all classes were added together to calculate the numbers of positive droplets and negative droplets in all droplets for each assumed total number of copies of the analyte present before emulsion formation. Thus, a table associating the number of copies of the analyte with the numbers of positive droplets and negative droplets was generated.

For each of Emulsions 2 to 4, a table was generated by the procedure described above. The tables generated for Emulsions 1 to 4 are shown in Table 5.

TABLE 5

| Number | | | | | | | | |
|---|---|---|---|---|---|---|---|---|
| of copies | Emulsion 1 | | Emulsion 2 | | Emulsion 3 | | Emulsion 4 | |
| of analyte | Negative | Positive | Negative | Positive | Negative | Positive | Negative | Positive |
| (copies) | (droplets) | (droplets) | (droplets) | (droplets) | (droplets) | (droplets) | (droplets) | (droplets) |
| 100,000 | 8 | 510 | 28 | 634 | 182 | 1,414 | 63 | 920 |
| 30,000 | 33 | 485 | 92 | 570 | 413 | 1,183 | 180 | 803 |
| 10,000 | 71 | 447 | 171 | 491 | 649 | 947 | 316 | 667 |
| 3,000 | 140 | 378 | 262 | 400 | 901 | 695 | 479 | 504 |
| 1,000 | 225 | 293 | 357 | 305 | 1,140 | 456 | 628 | 355 |
| 300 | 344 | 174 | 484 | 178 | 1,375 | 221 | 792 | 191 |
| 100 | 437 | 81 | 580 | 82 | 1,505 | 91 | 901 | 82 |
| 30 | 488 | 30 | 635 | 27 | 1,568 | 28 | 954 | 29 |
| 10 | 509 | 9 | 654 | 8 | 1,586 | 10 | 973 | 10 |
| 3 | 517 | 1 | 662 | 0 | 1,596 | 0 | 983 | 0 |

Concentration Calculation

As shown in Table 1, the total number of negative droplets for Emulsion 1 was 190 droplets. This was compared with the table shown in Table 5 to calculate the number of copies of the analyte in the sample. Specifically, in the table for Emulsion 1 in Table 5, the number of copies of the analyte for 140 negative droplets was 3,000 copies, and the number of copies of the analyte for 225 negative droplets was 1,000 copies. The detection results for Emulsion 1 showed that the number of negative droplets was 190 droplets; therefore, the number of copies of the analyte for 190 negative droplets was calculated by linear approximation between the above two points in the table. As a result, in this example, the number of copies of the analyte for Emulsion 1 was estimated to be 1,830 copies. This was divided by the total volume of all droplets subjected to detection. As a result, the concentration of the analyte in the reaction solution was calculated to be 7,970 copies/μL. By multiplication by a dilution factor of 10, the concentration of the analyte in the sample was calculated to be 79,700 copies/μL.

For each of Emulsions 2 to 4, calculations were performed in the same manner as for Emulsion 1 to calculate the concentration of the analyte in the sample. The calculation results are summarized in Table 6.

TABLE 6

| | | Total number of copies of | |
|---|---|---|---|
| | Relative | analyte present in all droplets | Concentration of |
| | dilution | subjected to detection | analyte in sample |
| Emulsion | factor | (copies) | (copies/μL) |
| Emulsion 1 | 1 | 1,830 | 79,700 |
| Emulsion 2 | 10 | 118 | 3,720 |
| Emulsion 3 | 100 | 26.2 | 382 |
| Emulsion 4 | 1,000 | 6.4 | 157 |

### Example 2

In Example 1, the total number of copies of the analyte present before emulsion formation (setting value) was roughly assumed as described above for table generation; in this example, finer setting values were assumed, and a more detailed table was generated and used to calculate the concentration of the analyte. Specifically, in this example, setting values were assumed at intervals of 1 copy, and calculations were performed as in Example 1 to generate a table.

A portion of the table generated for Emulsion 1 in which the number of copies of the analyte (setting value) ranges from 1,520 copies to 1,530 copies is shown in Table 7.

TABLE 7

Emulsion 1

| Number of copies of analyte | Negative | Positive |
|---|---|---|
| (copies) | (droplets) | (droplets) |
| 1,520 | 192 | 326 |
| 1,521 | 191 | 327 |
| 1,522 | 190 | 328 |
| 1,523 | 190 | 328 |
| 1,524 | 190 | 328 |
| 1,525 | 190 | 328 |
| 1,526 | 190 | 328 |
| 1,527 | 189 | 329 |
| 1,528 | 189 | 329 |
| 1,529 | 189 | 329 |
| 1,530 | 189 | 329 |

As shown in Table 1, the total number of negative droplets for Emulsion 1 was 190 droplets. This was compared with the partial table shown in Table 7 to calculate the number of copies of the analyte in the sample. In Table 7, the number of copies of the analyte for 190 negative droplets ranged from 1,522 copies to 1,526 copies. These were averaged to calculate the number of copies of the analyte for 190 negative droplets. As a result, in this example, the number of copies of the analyte for Emulsion 1 was estimated to be 1,524 copies. This was divided by the total volume of all droplets subjected to detection. As a result, the concentration of the analyte in the reaction solution was calculated to be 6,660 copies/μL. By multiplication by a dilution factor of 10, the concentration of the analyte in the sample was calculated to be 66,600 copies/μL.

A portion of the table generated for Emulsion 2 in which the number of copies of the analyte (setting value) ranges from 109 copies to 114 copies is shown in Table 8.

TABLE 8

Emulsion 2

| Number of copies of analyte | Negative | Positive |
|---|---|---|
| (copies) | (droplets) | (droplets) |
| 109 | 574 | 88 |
| 110 | 574 | 88 |
| 111 | 573 | 89 |
| 112 | 573 | 89 |
| 113 | 572 | 90 |
| 114 | 570 | 92 |

As shown in Table 1, the total number of negative droplets for Emulsion 2 was 573 droplets. This was compared with the partial table shown in Table 8 to calculate the number of copies of the analyte in the sample. In Table 8, the number of copies of the analyte for 573 negative droplets ranged from 111 copies to 112 copies. These were averaged to calculate the number of copies of the analyte for 573 negative droplets. As a result, in this example, the number of copies of the analyte for Emulsion 2 was estimated to be 112 copies. This was divided by the total volume of all droplets subjected to detection. As a result, the concentration of the analyte in the reaction solution was calculated to be 352 copies/μL. By multiplication by a dilution factor of 10, the concentration of the analyte in the sample was calculated to be 3,520 copies/μL.

A portion of the table generated for Emulsion 3 in which the number of copies of the analyte (setting value) ranges from 23 copies to 28 copies is shown in Table 9.

TABLE 9

Emulsion 3

| Number of copies of analyte | Negative | Positive |
|---|---|---|
| (copies) | (droplets) | (droplets) |
| 23 | 1,576 | 20 |
| 24 | 1,574 | 22 |
| 25 | 1,573 | 23 |
| 26 | 1,570 | 26 |
| 27 | 1,569 | 27 |
| 28 | 1.568 | 28 |

As shown in Table 1, the total number of negative droplets for Emulsion 3 was 1,571 droplets. This was compared with the partial table shown in Table 9 to calculate the number of copies of the analyte in the sample. In Table 9, the number of copies of the analyte for 1,573 negative droplets was 25 copies, and the number of copies of the analyte for 1,570 negative droplets was 26 copies. The detection results for Emulsion 3 showed that the number of negative droplets was 1,571 droplets; therefore, the number of copies of the analyte for 1,571 negative droplets was calculated by linear approximation between the above two points in the table. As a result, in this example, the number of copies of the analyte for Emulsion 3 was estimated to be 26 copies. This was divided by the total volume of all droplets subjected to detection. As a result, the concentration of the analyte in the reaction solution was calculated to be 38 copies/μL. By multiplication by a dilution factor of 10, the concentration of the analyte in the sample was calculated to be 380 copies/μL.

A portion of the table generated for Emulsion 4 in which the number of copies of the analyte (setting value) ranges from 3 copies to 8 copies is shown in Table 10.

TABLE 10

Emulsion 4

| Number of copies of analyte | Negative | Positive |
|---|---|---|
| (copies) | (droplets) | (droplets) |
| 3 | 983 | 0 |
| 4 | 983 | 0 |
| 5 | 982 | 1 |
| 6 | 978 | 5 |
| 7 | 975 | 8 |
| 8 | 974 | 9 |

As shown in Table 1, the total number of negative droplets for Emulsion 4 was 978 droplets. This was compared with the partial table shown in Table 10 to calculate the number of copies of the analyte in the sample. In Table 10, the number of copies of the analyte for 978 negative droplets was 6 copies. Thus, in this example, the number of copies of the analyte for Emulsion 4 was estimated to be 6 copies. This was divided by the total volume of all droplets subjected to detection. As a result, the concentration of the analyte in the reaction solution was calculated to be 14.7 copies/μL. By multiplication by a dilution factor of 10, the concentration of the analyte in the sample was calculated to be 147 copies/μL.

The above calculation results for Emulsions 1 to 4 are summarized in Table 11.

TABLE 11

| | | Total number of copies of | |
|---|---|---|---|
| | Relative | analyte present in all droplets | Concentration of |
| | dilution | subjected to detection | analyte in sample |
| Emulsion | factor | (copies) | (copies/μL) |
| Emulsion 1 | 1 | 1,524 | 66,600 |
| Emulsion 2 | 10 | 112 | 3,520 |
| Emulsion 3 | 100 | 26 | 380 |
| Emulsion 4 | 1,000 | 6 | 147 |

Comparison Between Comparative Example 1 and Examples 1 and 2

As shown in Tables 3, 6, and 11, in each of Comparative Example 1 and Examples 1 and 2, the dilution factors of Emulsions 1, 2, 3, and 4 relative to Emulsion 1 are 1-fold, 10-fold, 100-fold, and 1,000-fold, respectively. Hence, the concentrations of the analyte in the samples for Emulsions 1, 2, 3, and 4 should be 1 time, 0.1 times, 0.01 times, and 0.001 times, respectively, that for Emulsion 1.

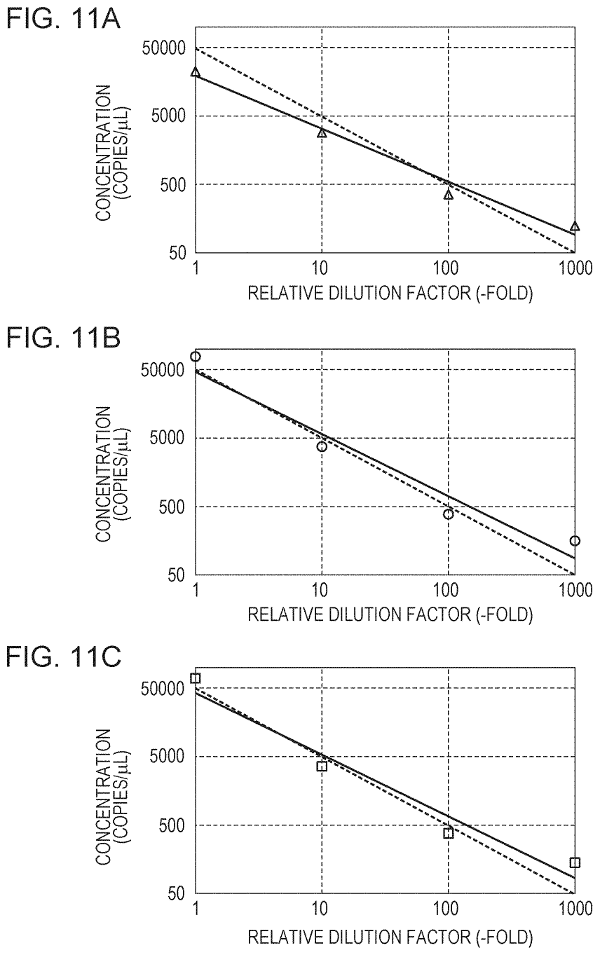

FIGS. 11A to 11C are graphs showing the relationship between the relative dilution factor and the calculation results of the concentrations of the analyte in the samples for Comparative Example 1 and Examples 1 and 2. FIGS. 11A, 11B, and 11C show the results for Comparative Example 1, Example 1, and Example 2, respectively, in the form of a log-log graph having a horizontal axis representing the relative dilution factor and a vertical axis representing the calculation results of the concentration.

As described above, the relative dilution factor and the concentration have the relationship y=ax⁻¹, where x is the relative dilution factor, and y is the concentration. Hence, their relationship should be expressed as a straight line with a gradient of −1 in a log-log graph. In FIGS. 11A to 11C, the dotted lines represent the relationship between the relative dilution factor and the concentration on the assumption that the concentration of the analyte in the sample for Emulsion 1 was 5×10⁴ copies/μL. In FIGS. 11A to 11C, the solid lines represent approximate curves obtained by power approximation of the results for Comparative Example 1 and Examples 1 and 2 in a log-log graph. A comparison between FIGS. 11A to 11C shows that the gradients of the solid lines in FIGS. 11B and 11C are closer to those of the dotted lines than that in FIG. 11A. Specifically, the gradient of the approximate curve for Comparative Example 1 was −0.77, the gradient of the approximate curve for Example 1 was −0.91, and the gradient of the approximate curve for Example 2 was −0.89. This demonstrates that the results for Examples 1 and 2 were closer to the true value than those for Comparative Example 1, that is, the reliability of quantitative analysis for Examples 1 and 2 was higher than that for Comparative Example 1. In particular, the results for Examples 1 and 2 were found to be closer to the true value at low dilution factors, that is, at high concentrations of the analyte in the sample. The above results demonstrate that, according to the present invention, analytical results with high reliability can be yielded even if there is variation in the size of droplets.

### Comparative Example 2

For each of Emulsions 5 to 8 of Preparation Examples 5 to 8, the concentration of the analyte in the sample was calculated as in Comparative Example 1. The results are shown in Table 12.

TABLE 12

| | | Total number of copies of | |
|---|---|---|---|
| | Relative | analyte present in all droplets | Concentration of |
| | dilution | subjected to detection | analyte in sample |
| Emulsion | factor | (copies) | (copies/μL) |
| Emulsion 5 | 1 | 659 | 16,800 |
| Emulsion 6 | 10 | 160 | 3,640 |
| Emulsion 7 | 100 | 10 | 294 |
| Emulsion 8 | 1000 | 7 | 151 |

### Example 3

For each of Emulsions 5 to 8, a table associating the numbers of positive droplets and negative droplets with the number of copies of the analyte was generated based on the size distribution of the droplets in the emulsion. The concentration of the analyte was calculated based on the table and the negative-positive information.

Table Generation

For each of Emulsions 5 to 8, a table (calibration curve) associating the numbers of positive droplets and negative droplets with the number of copies of the analyte was generated as in Example 1. The generated table is shown in Table 13.

TABLE 13

| Number | | | | | | | | |
|---|---|---|---|---|---|---|---|---|
| of copies | Emulsion 5 | | Emulsion 6 | | Emulsion 7 | | Emulsion 8 | |
| of analyte | Negative | Positive | Negative | Positive | Negative | Positive | Negative | Positive |
| (copies) | (droplets) | (droplets) | (droplets) | (droplets) | (droplets) | (droplets) | (droplets) | (droplets) |
| 100,000 | 62 | 779 | 82 | 901 | 51 | 765 | 23 | 787 |
| 30,000 | 164 | 677 | 206 | 777 | 157 | 659 | 66 | 744 |
| 10,000 | 266 | 575 | 329 | 654 | 282 | 534 | 147 | 663 |
| 3,000 | 384 | 457 | 474 | 509 | 414 | 402 | 286 | 524 |
| 1,000 | 509 | 332 | 623 | 360 | 525 | 291 | 442 | 368 |
| 300 | 656 | 185 | 790 | 193 | 646 | 170 | 612 | 198 |
| 100 | 757 | 84 | 897 | 86 | 735 | 81 | 724 | 86 |
| 30 | 815 | 26 | 953 | 30 | 789 | 27 | 782 | 28 |
| 10 | 830 | 11 | 973 | 10 | 807 | 9 | 800 | 10 |
| 3 | 841 | 0 | 983 | 0 | 816 | 0 | 810 | 0 |

Concentration Calculation

For each of Emulsions 5 to 8, the total number of negative droplets was compared with the table shown in Table 13 as in Example 1 to calculate the number of copies of the analyte in the sample. The calculation results are summarized in Table 14.

TABLE 14

| | | Total number of copies of | |
|---|---|---|---|
| | Relative | analyte present in all droplets | Concentration of |
| | dilution | subjected to detection | analyte in sample |
| Emulsion | factor | (copies) | (copies/μL) |
| Emulsion 5 | 1 | 3,000 | 76,500 |
| Emulsion 6 | 10 | 219 | 4,970 |
| Emulsion 7 | 100 | 11.3 | 334 |
| Emulsion 8 | 1,000 | 7.9 | 171 |

### Example 4

For each of Emulsions 5 to 8 of Preparation Examples 5 to 8, a table for correcting the concentration of the analyte calculated from the negative-positive information on the assumption that the size distribution of the reaction fields was monodisperse was generated based on the size distribution of the emulsion. The generated table was used to calculate the concentration of the analyte.

Table Generation

The size distributions of the droplets in Emulsions 5 to 8 after thermal cycling were as shown in Table 2. For each of Emulsions 5 to 8, a table (correction table) was generated based on the size distribution by the procedure described in the second embodiment.

A specific table generation procedure will now be described using Emulsion 5 as an example. As in Example 3, the total number of copies of the analyte present before emulsion formation (setting value) was assumed, and the numbers of positive droplets and negative droplets were calculated. The results are similar to those for Example 3 and are as shown in Table 13.

Next, based on the negative-positive information calculated for each setting value, the number of copies of the analyte and the concentration of the analyte in the sample (referred to as λ₂) were calculated on the assumption that the size distribution of the droplets in the emulsion was monodisperse. Here, as in Comparative Example 1, calculations were performed based on equation (4). Each setting value was divided by the total volume of the droplets in the emulsion and was then multiplied by a dilution factor of 10 to calculate the concentration of the analyte in the sample for the setting value (referred to as λ₁). In this example, the percent deviation of λ₂ from λ₁ ((λ₂−λ₁)/λ₁×100%) was calculated for each setting value. The results are shown in Table 15.

TABLE 15

| | | | | Calculated backwards from | | |
|---|---|---|---|---|---|---|
| | | (1) Negative-positive | | (1) on assumption that size | | |
| Setting value | | information calculated | | distribution was monodisperse | | |
| Number of | | based on size distribution | | Number of | | |
| copies of | Concentration | and Poisson distribution | | copies of | Concentration | Percent |
| analyte | λ₁ of analyte | Negative | Positive | analyte | λ₂ of analyte | deviation of |
| (copies) | (copies/μL) | (droplets) | (droplets) | (copies) | (copies/μL) | λ₂ from λ₁ |
| 100,000 | 2,600,000 | 62 | 779 | 2,190 | 56,000 | −98% |
| 30,000 | 770,000 | 164 | 677 | 1,380 | 35,000 | −95% |
| 10,000 | 260,000 | 266 | 575 | 968 | 25,000 | −90% |
| 3,000 | 77,000 | 384 | 457 | 659 | 17,000 | −78% |
| 1,000 | 26,000 | 509 | 332 | 422 | 11,000 | −59% |
| 300 | 7,700 | 656 | 185 | 209 | 5,300 | −30% |
| 100 | 2,600 | 757 | 84 | 88 | 2,300 | −12% |
| 30 | 770 | 815 | 26 | 26 | 660 | −13% |
| 10 | 260 | 830 | 11 | 11 | 280 | 10% |
| 3 | 77 | 841 | 0 | 0 | 0 | — |

Concentration Calculation

As shown in Table 12, the concentration (λ₂) of the analyte in the sample for Emulsion 5 calculated from the negative-positive information on the assumption that the size distribution of the reaction fields was monodisperse was 16,800 copies/μL. This was compared with the correction table (Table 15) to calculate the concentration of the analyte in the sample.

The percent deviation of the backwards-calculated concentration (λ₂) of the analyte for 16,800 copies/μL in Table 15 was first calculated to be −78% by linear approximation between two points at 11,000 copies/μL and 17,000 copies/μL. This percent deviation was used to correct the concentration λ₂ calculated from the negative-positive information for Emulsion 5 to calculate the concentration of the analyte in the sample as follows:

16,800/(1−0.78)=76,000 (copies/μL)

For each of Emulsions 6 to 8, a correction table was similarly generated and used to calculate the concentration of the analyte in the sample. The correction tables for Emulsions 6 to 8 are shown in Tables 16 to 18. The calculation results of the concentrations of the analyte in the samples for Emulsions 5 to 8 are summarized in Table 19.

TABLE 16

| | | | | Calculated backwards from | | |
|---|---|---|---|---|---|---|
| | | (1) Negative-positive | | (1) on assumption that size | | |
| Setting value | | information calculated | | distribution was monodisperse | | |
| Number of | | based on size distribution | | Number of | | |
| copies of | Concentration | and Poisson distribution | | copies of | Concentration | Percent |
| analyte | λ₁ of analyte | Negative | Positive | analyte | λ₂ of analyte | deviation of |
| (copies) | (copies/μL) | (droplets) | (droplets) | (copies) | (copies/μL) | λ₂ from λ₁ |
| 100,000 | 2,300,000 | 82 | 901 | 2,440 | 56,000 | −98% |
| 30,000 | 680,000 | 206 | 777 | 1,540 | 35,000 | −95% |
| 10,000 | 230,000 | 329 | 654 | 1,080 | 24,000 | −89% |
| 3,000 | 68,000 | 474 | 509 | 717 | 16,000 | −76% |
| 1,000 | 23,000 | 623 | 360 | 448 | 10,000 | −55% |
| 300 | 6,800 | 790 | 193 | 215 | 4,900 | −28% |
| 100 | 2,300 | 897 | 86 | 90 | 2,100 | −10% |
| 30 | 680 | 953 | 30 | 30 | 680 | 0% |
| 10 | 230 | 973 | 10 | 10 | 230 | 0% |
| 3 | 68 | 983 | 0 | 0 | 0 | — |

TABLE 17

| | | | | Calculated backwards from | | |
|---|---|---|---|---|---|---|
| | | (1) Negative-positive | | (1) on assumption that size | | |
| Setting value | | information calculated | | distribution was monodisperse | | |
| Number of | | based on size distribution | | Number of | | |
| copies of | Concentration | and Poisson distribution | | copies of | Concentration | Percent |
| analyte | λ₁ of analyte | Negative | Positive | analyte | λ₂ of analyte | deviation of |
| (copies) | (copies/μL) | (droplets) | (droplets) | (copies) | (copies/μL) | λ₂ from λ₁ |
| 100,000 | 2,900,000 | 51 | 765 | 2,260 | 67,000 | −98% |
| 30,000 | 880,000 | 157 | 659 | 1,350 | 40,000 | −96% |
| 10,000 | 290,000 | 282 | 534 | 867 | 26,000 | −91% |
| 3,000 | 88,000 | 414 | 402 | 554 | 16,000 | −82% |
| 1,000 | 29,000 | 525 | 291 | 360 | 11,000 | −64% |
| 300 | 8,800 | 646 | 170 | 191 | 5,600 | −36% |
| 100 | 2,900 | 735 | 81 | 85 | 2,500 | −15% |
| 30 | 880 | 789 | 27 | 27 | 800 | −10% |
| 10 | 290 | 807 | 9 | 9 | 270 | −10% |
| 3 | 88 | 816 | 0 | 0 | 0 | — |

TABLE 18

| | | | | Calculated backwards from | | |
|---|---|---|---|---|---|---|
| | | (1) Negative-positive | | (1) on assumption that size | | |
| Setting value | | information calculated | | distribution was monodisperse | | |
| Number of | | based on size distribution | | Number of | | |
| copies of | Concentration | and Poisson distribution | | copies of | Concentration | Percent |
| analyte | λ₁ of analyte | Negative | Positive | analyte | λ₂ of analyte | deviation of |
| (copies) | (copies/μL) | (droplets) | (droplets) | (copies) | (copies/μL) | λ₂ from λ₁ |
| 100,000 | 2,200,000 | 23 | 787 | 2885 | 62000 | −97% |
| 30,000 | 650,000 | 66 | 744 | 2031 | 44000 | −93% |
| 10,000 | 220,000 | 147 | 663 | 1382 | 30000 | −86% |
| 3,000 | 65,000 | 286 | 524 | 843 | 18000 | −72% |
| 1,000 | 22,000 | 442 | 368 | 491 | 11000 | −51% |
| 300 | 6,500 | 612 | 198 | 227 | 4900 | −24% |
| 100 | 2,200 | 724 | 86 | 91 | 2000 | −9% |
| 30 | 650 | 782 | 28 | 28 | 600 | −7% |
| 10 | 220 | 800 | 10 | 10 | 220 | 0% |
| 3 | 65 | 810 | 0 | 0 | 0 | — |

TABLE 19

| | Relative | Concentration of |
|---|---|---|
| | dilution | analyte in sample |
| Emulsion | factor | (copies/μL) |
| Emulsion 5 | 1 | 76,000 |
| Emulsion 6 | 10 | 4,550 |
| Emulsion 7 | 100 | 328 |
| Emulsion 8 | 1,000 | 151 |

Comparison Between Comparative Example 2 and Examples 3 and 4

As shown in Tables 12, 14, and 19, in each of Comparative Example 2 and Examples 3 and 4, the dilution factors of Emulsions 5, 6, 7, and 8 relative to Emulsion 5 are 1-fold, 10-fold, 100-fold, and 1,000-fold, respectively. Hence, the concentrations of the analyte in the samples for Emulsions 5, 6, 7, and 8 should be 1 time, 0.1 times, 0.01 times, and 0.001 times, respectively, that for Emulsion 5.

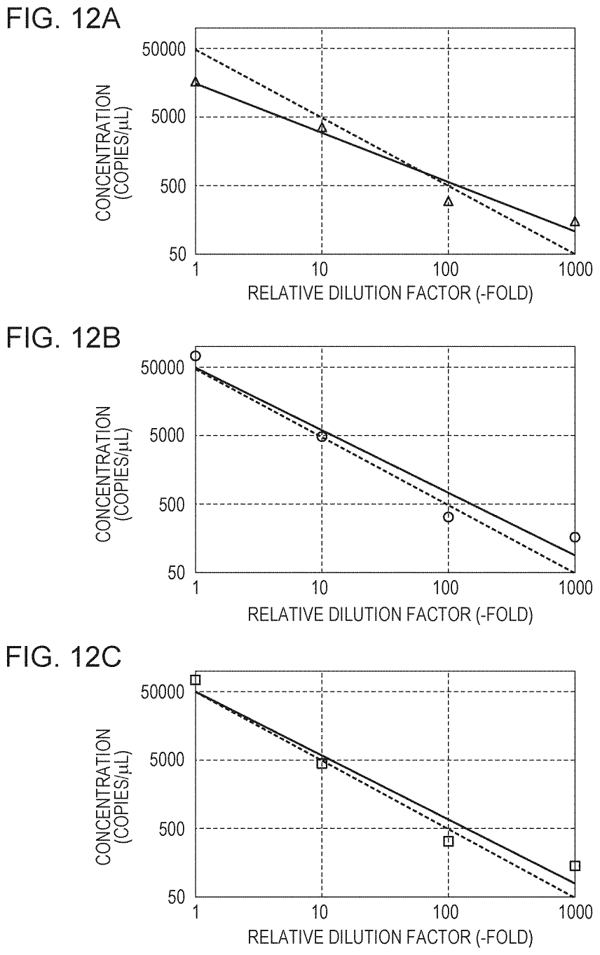

FIGS. 12A to 12C are graphs showing the relationship between the relative dilution factor and the calculation results of the concentrations of the analyte in the samples for Comparative Example 2 and Examples 3 and 4. FIGS. 12A, 12B, and 12C show the results for Comparative Example 2, Example 3, and Example 4, respectively, in the form of a log-log graph having a horizontal axis representing the relative dilution factor and a vertical axis representing the calculation results of the concentration.

As described above, the relative dilution factor and the concentration have the relationship y=ax⁻¹, where x is the relative dilution factor, and y is the concentration. Hence, their relationship should be expressed as a straight line with a gradient of −1 in a log-log graph. In FIGS. 12A to 12C, the dotted lines represent the relationship between the relative dilution factor and the concentration on the assumption that the concentration of the analyte in the sample for Emulsion 5 was 5×10⁴ copies/μL. In FIGS. 12A to 11C, the solid lines represent approximate curves obtained by power approximation of the results for Comparative Example 2 and Examples 3 and 4 in a log-log graph. A comparison between FIGS. 12A to 12C shows that the gradients of the solid lines in FIGS. 12B and 12C are closer to those of the dotted lines than that in FIG. 12A. Specifically, the gradient of the approximate curve for Comparative Example 2 was −0.77, the gradient of the approximate curve for Example 3 was −0.91, and the gradient of the approximate curve for Example 4 was −0.93. This demonstrates that the results for Examples 3 and 4 were closer to the true value than those for Comparative Example 2, that is, the reliability of quantitative analysis for Examples 3 and 4 was higher than that for Comparative Example 2. In particular, the results for Examples 3 and 4 were found to be closer to the true value at low dilution factors, that is, at high concentrations of the analyte in the sample. The above results demonstrate that, according to the present invention, analytical results with high reliability can be yielded even if there is variation in the size of droplets.

According to the present invention, the reliability of analytical results can be improved in a simpler manner than in the known art even if there is variation in the size of reaction fields.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An analysis system for analyzing a concentration of an analyte in a sample, the analysis system comprising:
an analyte-information acquiring section configured to acquire at least one piece of information about a plurality of reaction fields generated by splitting a liquid containing the sample, the at least one piece of information being selected from the group consisting of information about a number of positive reaction fields that are reaction fields in which the analyte is detected and information about a number of negative reaction fields that are reaction fields in which no analyte is detected; and
a determining section configured to determine a number of molecules or particles or concentration of the analyte in the sample based on
the at least one piece of information acquired by the analyte-information acquiring section, the at least one piece of information being selected from the group consisting of the information about the number of positive reaction fields and the information about the number of negative reaction fields, and
a table associating the at least one piece of information selected from the group consisting of the information about the number of positive reaction fields and the information about the number of negative reaction fields with the number of molecules or particles or concentration of the analyte in the sample or in at least some of the plurality of reaction fields.

2. The analysis system according to claim 1, wherein the table is generated based on information about a size of each of the plurality of reaction fields.

3. The analysis system according to claim 1, wherein the analyte-information acquiring section is configured to detect the analyte in the plurality of reaction fields.

4. The analysis system according to claim 1, further comprising:
a size-information acquiring section configured to acquire information about a size of each of the plurality of reaction fields; and
a table-generating section configured to generate the table based on the size information acquired by the size-information acquiring section.

5. The analysis system according to claim 4, wherein the table-generating section is configured to generate the table based on the size information acquired by the size-information acquiring section by assuming the number of molecules or particles of the analyte and performing a simulation based on a Poisson model.

6. The analysis system according to claim 4, further comprising:
a storage section configured to store a plurality of tables; and
a table-selecting section configured to select the table used for concentration determination from the plurality of tables stored in the storage section based on size information different from the size information acquired by the size-information acquiring section and used for table generation.

7. The analysis system according to claim 1, further comprising:
a size-information acquiring section configured to acquire information about a size of each of the plurality of reaction fields;
a storage section configured to store a plurality of tables; and
a table-selecting section configured to select the table used for concentration determination from the plurality of tables stored in the storage section based on the size information acquired by the size-information acquiring section.

8. The analysis system according to claim 4, wherein the size-information acquiring section and the analyte-information acquiring section each include an image capture unit configured to capture an image of at least some of the plurality of reaction fields.

9. The analysis system according to claim 1, wherein
the at least one piece of information selected from the group consisting of the information about the number of positive reaction fields and the information about the number of negative reaction fields in the table is discrete, and
the determining section is configured to determine the number of molecules or particles or concentration of the analyte in the sample by interpolating the at least one piece of information selected from the group consisting of the information about the number of positive reaction fields and the information about the number of negative reaction fields in the table.

10. The analysis system according to claim 1, further comprising a reaction-field generating section configured to split the liquid to generate the plurality of reaction fields.

11. The analysis system according to claim 10, wherein the reaction-field generating section is configured to generate an emulsion of the liquid dispersed in droplet form in a second liquid incompatible with the liquid.

12. The analysis system according to claim 11, wherein the reaction-field generating section is configured to generate the emulsion by membrane emulsification or mechanical emulsification.

13. The analysis system according to claim 1, wherein the liquid contains a chemical for making the analyte detectable,
the analysis system further comprising a reaction section configured to allow a reaction due to the chemical to proceed in each of the plurality of reaction fields to make the analyte detectable.

14. The analysis system according to claim 1, wherein the analyte comprises a nucleic acid.

15. The analysis system according to claim 14, wherein the chemical comprises an amplification reagent for amplification of the nucleic acid and a fluorescent reagent that emits fluorescence by interaction with the nucleic acid.

16. The analysis system according to claim 14, wherein the reaction comprises PCR.

17. The analysis system according to claim 13, wherein the reaction section includes a temperature regulator configured to regulate a temperature of each of the plurality of reaction fields.

18. The analysis system according to claim 1, wherein a size distribution of the plurality of reaction fields is polydisperse.

19. An analysis system for analyzing a concentration of an analyte in a sample, the analysis system comprising:
an analyte-information acquiring section configured to acquire at least one piece of information about a plurality of reaction fields which are generated by splitting a liquid containing the sample and whose size distribution is polydisperse, the at least one piece of information being selected from the group consisting of information about a number of positive reaction fields that are reaction fields in which the analyte is detected and information about a number of negative reaction fields that are reaction fields in which no analyte is detected; and
a determining section configured to determine a number of molecules or particles or concentration of the analyte in the sample or in at least some of the plurality of reaction fields on an assumption that the size distribution of the plurality of reaction fields is monodisperse, based on the at least one piece of information acquired by the analyte-information acquiring section, the at least one piece of information being selected from the group consisting of the information about the number of positive reaction fields and the information about the number of negative reaction fields; and
a correcting section configured to correct the number of molecules or particles or concentration of the analyte in the sample or in the at least some of the plurality of reaction fields determined by the determining section on the assumption that the size distribution of the plurality of reaction fields is monodisperse, based on a table for correcting, depending on the size distribution of the plurality of reaction fields, the number of molecules or particles or concentration of the analyte in the sample or in the at least some of the plurality of reaction fields.

20. The analysis system according to claim 19, wherein the table associates
the number of molecules or particles or concentration of the analyte in the sample or in the at least some of the plurality of reaction fields, with
a number of molecules or particles or concentration of the analyte in the sample or in the at least some of the plurality of reaction fields determined on an assumption that the size distribution of the plurality of reaction fields is monodisperse from at least one piece of information selected from the group consisting of information about the number of positive reaction fields and information about the number of negative reaction fields probabilistically estimated based on the number of molecules or particles or concentration of the analyte and the size distribution of the plurality of reaction fields.

21. An analysis method for analyzing a concentration of an analyte in a sample, the analysis method comprising:
an analyte-information acquiring step of acquiring at least one piece of information about a plurality of reaction fields generated by splitting a liquid containing the sample, the at least one piece of information being selected from the group consisting of information about a number of positive reaction fields that are reaction fields in which the analyte has been detected and information about a number of negative reaction fields that are reaction fields in which no analyte has been detected; and
a determining step of determining a number of molecules or particles or concentration of the analyte in the sample based on
the at least one piece of information acquired by the analyte-information acquiring step, the at least one piece of information being selected from the group consisting of the information about the number of positive reaction fields and the information about the number of negative reaction fields, and
a table associating the at least one piece of information selected from the group consisting of the information about the number of positive reaction fields and the information about the number of negative reaction fields with the number of molecules or particles or concentration of the analyte in the sample or in at least some of the plurality of reaction fields.

22. A computer-readable storage medium storing a program configured to cause a computer to perform a process on detection data containing at least one piece of information about a plurality of reaction fields generated by splitting a liquid containing a sample containing an analyte, the at least one piece of information being selected from the group consisting of information about a number of positive reaction fields that are reaction fields in which the analyte has been detected and information about a number of negative reaction fields that are reaction fields in which no analyte has been detected,
the process comprising:
a determining step of determining a number of molecules or particles or concentration of the analyte in the sample based on
the at least one piece of information selected from the group consisting of the information about the number of positive reaction fields and the information about the number of negative reaction fields, and
a table associating the at least one piece of information selected from the group consisting of the information about the number of positive reaction fields and the information about the number of negative reaction fields with the number of molecules or particles or concentration of the analyte in the sample or in at least some of the plurality of reaction fields.

23. An analysis method for analyzing a concentration of an analyte in a sample, the analysis method comprising:
an analyte-information acquiring step of acquiring at least one piece of information about a plurality of reaction fields which are generated by splitting a liquid containing the sample and whose size distribution is polydisperse, the at least one piece of information being selected from the group consisting of information about a number of positive reaction fields that are reaction fields in which the analyte has been detected and information about a number of negative reaction fields that are reaction fields in which no analyte has been detected; and
a determining step of determining a number of molecules or particles or concentration of the analyte in the sample on an assumption that the size distribution of the plurality of reaction fields is monodisperse, based on the at least one piece of information acquired by the analyte-information acquiring step, the at least one piece of information being selected from the group consisting of the information about the number of positive reaction fields and the information about the number of negative reaction fields; and
a correcting step of correcting, depending on the size distribution of the plurality of reaction fields, the number of molecules or particles or concentration of the analyte in the sample determined by the determining step.

24. A computer-readable storage medium storing a program configured to cause a computer to perform a process on detection data containing at least one piece of information about a plurality of reaction fields which are generated by splitting a liquid containing a sample containing an analyte and whose size distribution is polydisperse, the at least one piece of information being selected from the group consisting of information about a number of positive reaction fields that are reaction fields in which the analyte has been detected and information about a number of negative reaction fields that are reaction fields in which no analyte has been detected,
the process comprising:
a determining step of determining a number of molecules or particles or concentration of the analyte in the sample on an assumption that the size distribution of the plurality of reaction fields is monodisperse, based on the at least one piece of information selected from the group consisting of the information about the number of positive reaction fields and the information about the number of negative reaction fields; and
a correcting step of correcting, depending on the size distribution of the plurality of reaction fields, the number of molecules or particles or concentration of the analyte in the sample determined by the determining step.
